(19) United States
(12) Patent Application Publication
(10) Pub. No.: US 2009/0099192 A1
(43) Pub. Date: Apr. 16, 2009

(54) Substituted 1H-pyrrole-2,5-diones

(75) Inventors: Thomas Bretschneider, Lohmar (DE); Mazen Es-Sayed, Langenfeld (DE); Reiner Fischer, Monheim (DE); Kristian Kather, Langenfeld (DE); Olga Malsam, Rosrath (DE); Jorg Konze, Koln (DE); Peter Losel, Leverkusen (DE); Erich Sanwald, Kiel (DE); Waltraud Hempel, Liederbach (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 11/632,885

(22) PCT Filed: Jul. 8, 2005
(86) PCT No.: PCT/EP2005/007416

§ 371 Date: Jul. 11, 2008

(30) Foreign Application Priority Data
Jul. 19, 2004 (DE) 10 2004 034 867.7

(51) Int. Cl. A01N 43/58; A01N 43/36; C07D 403/04; A01P 15/00; A01P 17/00; C07D 207/456

(52) U.S. Cl. 514/252.05; 514/425; 548/544; 544/238

(57) ABSTRACT

The invention relates to novel substituted 1H-pyrrole-2,5-diones of the formula (IA)

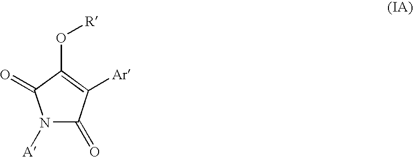

in which A′, Ar′ and R′ are as defined in the description, to novel compositions for controlling animal pests based on substituted 1H-pyrrole-2,5-diones of the formulae (I) and (IA)

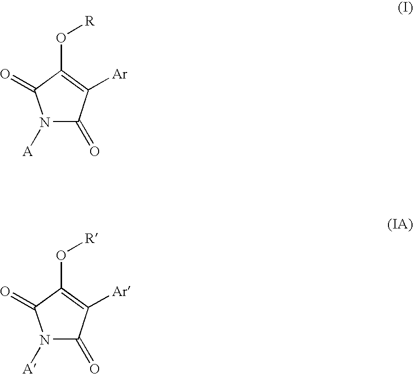

in which A, A′, Ar, Ar′, R and R′ are as defined in the description, and to processes for their preparation.

The invention relates to novel substituted 1H-pyrrole-2,5-diones, to their use for controlling animal pests, in particular insects, arachnids and nematodes, and to processes for their preparation, and also to the use of known substituted 1H-pyrrole-2,5-diones for controlling animal pests.

A number of substituted 1H-pyrrole-2,5-diones is already known from the literature (cf., for example, J. Am. Chem. Soc. 78 (1956), 4656-4659; J. Indian Chem. Soc. 49 (1972), 403-406; J. Med. Chem. 26 (1983), 700-714; J. Molec. Catal B: Enzymatic 1 (1996), 103-107).

However, the compounds given in the cited scientific journals are only described as products of chemical scientific work and without any relation to a use against animal pests.

It has now been found that the substituted 1H-pyrrole-2,5-diones, some of which are known, of the formula (I)

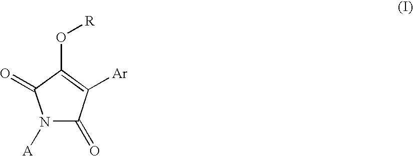

in which
A represents in each case optionally substituted alkyl, alkenyl, alkynyl, cycloalkyl, cycloalkenyl, cycloalkylalkyl, aryl, arylalkyl, heterocyclyl or heterocyclylalkyl,
A furthermore represents adamantyl,
Ar represents substituted aryl, and
R represents hydrogen or one of the groupings below

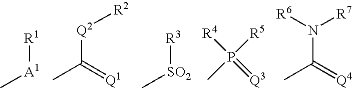

where
A¹ represents a carbonyl group (C═O) or represents methylene,
Q¹, Q², Q³ and Q⁴ each represent O (oxygen) or S (sulfur),
R¹ represents in each case optionally substituted alkyl, alkenyl, alkynyl, cycloalkyl, cycloalkylalkyl, aryl, arylalkyl, heterocyclyl or heterocyclylalkyl and, if A¹ represents methylene, also represents in each case optionally substituted alkoxy, alkylthio, alkylsulfinyl, alkylsulfonyl, alkylamino, dialkylamino, alkylcarbonylamino, N-alkyl-alkylcarbonylamino, alkoxycarbonylamino, N-alkyl-alkoxycarbonylamino, alkenyloxy, alkynyloxy, alkenylamino, alkynylamino, N-alkyl-alkenylamino, N-alkyl-alkynylamino, cycloalkyloxy, cycloalkylalkoxy, cycloalkylamino, cycloalkylalkylamino, N-alkyl-cycloalkylamino, N-alkyl-cycloalkylalkylamino, aryloxy, arylalkoxy, arylamino, arylalkylamino, N-alkyl-arylamino or N-alkyl-arylalkylamino,
R² represents in each case optionally substituted alkyl, alkenyl, alkynyl, cycloalkyl, cycloalkylalkyl, aryl, arylalkyl, heterocyclyl or heterocyclylalkyl,
R³ represents in each case optionally substituted alkyl, alkenyl, alkynyl, cycloalkyl, cycloalkylalkyl, aryl, arylalkyl, heterocyclyl or heterocyclylalkyl,
R⁴ and R⁵ are identical or different and independently of one another represent in each case optionally substituted alkyl, alkoxy, alkylthio, alkylamino, dialkylamino, phenyl, phenoxy or phenylthio,
R⁶ represents hydrogen or represents in each case optionally substituted alkyl or alkenyl, and
R⁷ represents hydrogen or represents in each case optionally substituted alkyl, alkoxy, alkenyl, cycloalkyl, cycloalkylalkyl, aryl or arylalkyl, or together with R⁶ represents optionally substituted alkanediyl in which optionally one methylene grouping is replaced by oxygen or sulfur,
   and salt-like derivatives of the compounds of the formula (I) resulting from reactions with basic compounds,
   are highly suitable for controlling animal pests in agriculture and in other areas of use.

Depending on the nature of the substituents, the compounds of the formula (I) may be present as geometrical and/or as optical isomers or corresponding isomer mixtures of varying composition. Both the pure isomers and the isomer mixtures can be used according to the invention.

Saturated or unsaturated hydrocarbon radicals, such as alkyl or alkenyl, can in each case be straight-chain or branched as far as this is possible, including in combination with heteroatoms, such as in alkoxy.

Optionally substituted radicals may be mono- or polysubstituted, where in the case of polysubstitution the substituents may be identical or different.

Halogen-substituted radicals, such as, for example, haloalkyl, may contain one or more halogen substituents. In the case of multiple substitution by halogen, these substituents can be identical or different. Here, halogen represents fluorine, chlorine, bromine or iodine, in particular fluorine, chlorine or bromine.

Preferred substituents or preferred ranges of the radicals present in the formula (I) given above are defined below.
A preferably represents optionally cyano-, halogen-, C₁-C₆-alkoxy-, C₁-C₆-alkylthio-, C₁-C₆-alkylsulfinyl-, C₁-C₆-alkylsulfonyl- or C₁-C₆-alkoxy-carbonyl-substituted C₁-C₈-alkyl, represents in each case optionally cyano- or halogen-substituted C₂-C₈-alkenyl or C₂-C₈-alkynyl, represents in each case optionally cyano-, halogen-, C₁-C₆-alkyl-, C₁-C₆-haloalkyl-, C₁-C₆-alkoxy- or C₁-C₆-haloalkoxy-substituted C₃-C₈-cycloalkyl, C₄-C₈-cycloalkenyl or C₃-C₈-cycloalkyl-C₁-C₄-alkyl, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl or phenyl-C₁-C₄-alkyl, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl- or C₁-C₃-alkylenedioxy-substituted heterocyclyl or heterocyclyl-C₁-C₄-alkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping.
A furthermore preferably represents in each case C₁-C₆-alkoxy-carbonyl-substituted C₃-C₈-cycloalkyl, C₄-C₈-cycloalkenyl, C₃-C₈-cycloalkyl-C₁-C₄-alkyl or adamantyl or represents optionally cyano-, halogen-, C₁-C₆-alkyl-, C₁-C₆-haloalkyl-, C₁-C₆-alkoxy- or C₁-C₆-haloalkoxy-substituted adamantyl.
A particularly preferably represents optionally cyano-, halogen-, C₁-C₅-alkoxy-, C₁-C₅-alkylthio-, C₁-C₅-alkylsulfinyl-, C₁-C₅-alkylsulfonyl- or C₁-C₅-alkoxy-carbonyl-substituted C₁-C₆-alkyl, represents in each case optionally cyano- or halogen-substituted C₂-C₆-alkenyl or C₂-C₆-alkynyl, represents in each case optionally cyano-, halogen-, C₁-C₄-alkyl-, C₁-C₄-haloalkyl-, C₁-C₄-alkoxy- or C₁-C₄-haloalkoxy-substituted C₃-C₇-cycloalkyl, C₅-C₇-cycloalkenyl or C₃-C₇-cycloalkyl-C₁-C₃-alkyl, represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted phenyl or phenyl-C₁-C₃-alkyl, or represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl- or C₁-C₂-alkylenedioxy-substituted heterocyclyl or heterocyclyl-C₁-C₄-alkyl, where the heterocyclyl grouping in each case contains 1 to 5 carbon atoms and 1 to 3 nitrogen atoms and/or 1 oxygen atom and/or 1 sulfur atom and/or one (SO) grouping and/or one (SO₂) grouping and optionally additionally one (C═O) grouping and/or one (C═S) grouping.
A furthermore particularly preferably represents in each case C₁-C₆-alkoxy-carbonyl-substituted C₃-C₇-cycloalkyl, C₅-C₇-cycloalkenyl, C₃-C₇-cycloalkyl-C₁-C₃-alkyl or adamantyl or represents optionally cyano-, halogen-, C₁-C₄-alkyl-, C₁-C₄-haloalkyl-, C₁-C₄-alkoxy- or C₁-C₄-haloalkoxy-substituted adamantyl.
A very particularly preferably represents in each case optionally cyano-, fluorine-, chlorine-, bromine-, methoxy-, ethoxy-, n- or i-propoxy-, n-, i-, s- or t-butoxy-, methylthio-, ethylthio-, n- or i-propylthio-, n-, i-, s- or t-butylthio-, methylsulfinyl-, ethylsulfinyl-, n- or i-propyl-sulfinyl-, methylsulfonyl-, ethylsulfonyl-, methoxycarbonyl-, ethoxycarbonyl-, n- or i-propoxycarbonyl-substituted methyl, ethyl, n- or i-propyl, n-, i-, s- or t-butyl, represents in each case optionally cyano-, fluorine-, chlorine- or bromine-substituted ethenyl, propenyl, butenyl, ethynyl, propynyl or butynyl, represents in each case optionally cyano-, fluorine-, chlorine-, bromine-, methyl-, ethyl-, n- or i-propyl-, methoxy- or ethoxy-substituted cyclopropyl, cyclobutyl, cyclopentyl, cyclohexyl, cyclobutenyl, cyclopentenyl, cyclohexenyl, cyclopropylmethyl, cyclobutylmethyl, cyclopentylmethyl or cyclohexylmethyl, represents in each case optionally nitro-, cyano-, fluorine-, chlorine-, bromine-, iodine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, difluoroethoxy-, trifluoroethoxy-, methylthio-, ethylthio-, n- or i-propylthio-, n-, i-, s- or t-butylthio-, difluoromethylthio-, trifluoromethylthio-, chlorodifluoromethylthio-, methoxycarbonyl-, ethoxycarbonyl-, n- or i-propoxycarbonyl-, n-, i-, s- or t-butoxycarbonyl-, methylenedioxy-, dimethylenedioxy-, difluoromethylenedioxy-, difluorodimethylenedioxy-, trifluorodimethylenedioxy- or chlorodifluorodimethylenedioxy-substituted phenyl, benzyl, phenylethyl or 1,1-dimethylbenzyl, or represents in each case optionally nitro-, cyano-, fluorine-, chlorine-, bromine-, iodine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, difluoroethoxy-, trifluoroethoxy-, methylthio-, ethylthio-, n- or i-propylthio-, n-, i-, s- or t-butylthio-, difluoromethylthio-, trifluoromethylthio-, chlorodifluoromethylthio-, methoxycarbonyl-, ethoxycarbonyl-, n- or i-propoxycarbonyl-, n-, i-, s- or t-butoxycarbonyl-, methylenedioxy-, dimethylenedioxy-, difluoromethylenedioxy-, difluorodimethylenedioxy-, trifluorodimethylenedioxy- or chlorodifluorodimethylenedioxy-substituted heterocyclyl, heterocyclylmethyl or heterocyclylethyl, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, piperidinyl, pyridinyl, pyrimidinyl, pyrazinyl, pyridazinyl.
A furthermore very particularly preferably represents in each case optionally cyano-, fluorine-, chlorine-, bromine-, methoxy-, ethoxy-, n- or i-propoxy-, n-, i-, s- or t-butoxy-, methylthio-, ethylthio-, n- or i-propylthio-, n-, i-, s- or t-butylthio-, methylsulfinyl-, ethylsulfinyl-, n- or i-propylsulfinyl-, methylsulfonyl-, ethylsulfonyl-, methoxycarbonyl-, ethoxycarbonyl-, n- or i-propoxycarbonyl-substituted n-, i-, s-, t- or neo-pentyl, represents in each case optionally cyano-, fluorine-, chlorine-, bromine-, methyl-, ethyl-, n- or i-propyl-, methoxy- or ethoxy-substituted adamantyl, represents in each case optionally n- or i-propoxy-, n-, i-, s- or t-butoxy-, methoxycarbonyl- or ethoxycarbonyl-, trifluoromethyl- or trifluoromethoxy-substituted cyclopropyl, cyclobutyl, cyclopentyl, cyclohexyl, cyclobutenyl, cyclopentenyl, cyclohexenyl, cyclopropylmethyl, cyclobutylmethyl, cyclopentylmethyl, cyclohexylmethyl or adamantyl, represents optionally cyano-, fluorine-, chlorine-, bromine-, methyl-, ethyl-, n- or i-propyl-substituted adamantyl or represents optionally nitro-, cyano-, fluorine-, chlorine-, bromine-, iodine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoro-methoxy-, chlorodifluoromethoxy-, fluoroethoxy-, difluoroethoxy-, trifluoroethoxy-, methylthio-, ethylthio-, n- or i-propylthio-, n-, i-, s- or t-butylthio-, difluoromethylthio-, trifluoromethylthio-, chlorodifluoromethylthio-, methoxycarbonyl-, ethoxycarbonyl-, n- or i-propoxycarbonyl-, n-, i, s- or t-butoxycarbonyl-, methylenedioxy-, dimethylenedioxy-, difluoromethylenedioxy-, difluorodimethylenedioxy-, trifluorodimethylenedioxy- or chlorodifluorodimethylenedioxy-substituted 1-methylbenzyl.
A especially preferably represents in each case optionally fluorine- or ethoxycarbonyl-substituted ethyl, n-propyl, i-propyl, t-butyl or propenyl, represents phenyl, benzyl or 1,1-dimethylbenzyl, each of which is optionally mono- to trisubstituted by fluorine, chlorine, methyl, trifluoromethyl, ethoxy or trifluoromethoxy, represents heterocyclyl, each of which is optionally substituted by trifluoromethyl or ethoxy, where the heterocyclyl grouping is in each case selected from the group consisting of thiazolyl and pyridazinyl, or represents the cyclohexyl grouping below

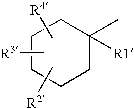

in which
R^{1′} represents hydrogen, cyano, methoxycarbonyl or ethoxycarbonyl,
R^{2′}, R^{3′} and R^{4′} are identical or different and individually represent hydrogen, methyl, i-propyl or methoxy.
A furthermore especially preferably represents pyranyl, cyclopropylmethyl, neo-pentyl, represents adamantyl, represents t-butoxycarbonyl-substituted piperidinyl or represents the cyclohexyl grouping below

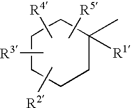

in which
R^{1′} represents hydrogen, methoxycarbonyl or ethoxycarbonyl,
R^{2′}, R^{3′}, R^{4′} and R^{5′} are identical or different and individually represent hydrogen, trifluoromethyl, methoxycarbonyl, ethoxycarbonyl or n-butoxy,
Ar preferably represents phenyl which is mono- to pentasubstituted by identical or different substituents, where the substituents are preferably taken from the list below: nitro, cyano, carbamoyl, thiocarbamoyl, halogen, C₁-C₆-alkyl, C₁-C₆-haloalkyl, C₁-C₆-alkoxy, C₁-C₆-haloalkoxy, C₁-C₄-alkoxy-C₁-C₄-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, phenyl, phenoxy, phenylthio, phenyl-C₁-C₄-alkyl, phenyl-C₁-C₄-alkoxy, phenyl-C₁-C₄-alkylthio (where the phenyl groups of the substituents each optionally contain one or more of the substituents listed above), heterocyclyl, heterocyclyloxy, heterocyclylthio, heterocyclyl-C₁-C₄-alkyl, heterocyclyl-C₁-C₄-alkoxy, heterocyclyl-C₁-C₄-alkylthio, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping,
Ar particularly preferably represents phenyl which is mono- to tetrasubstituted by identical or different substituents, where the substituents are preferably taken from the list below:
   nitro, cyano, carbamoyl, thiocarbamoyl, halogen, C₁-C₅-alkyl, C₁-C₅-haloalkyl, C₁-C₅-alkoxy, C₁-C₅-haloalkoxy, C₁-C₄-alkoxy-C₁-C₄-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, phenyl, phenoxy, phenylthio, phenyl-C₁-C₃-alkyl, phenyl-C₁-C₄-alkoxy, phenyl-C₁-C₃-alkylthio (where in each case the phenyl groups of the substituents optionally contain one or more of the substituents listed above), heterocyclyl, heterocyclyloxy, heterocyclylthio, heterocyclyl-C₁-C₃-alkyl, heterocyclyl-C₁-C₃-alkoxy, heterocyclyl-C₁-C₃-alkylthio, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping,
Ar very particularly preferably represents phenyl which is mono- to trisubstituted by identical or different substituents, where the substituents are preferably taken from the list below:
   nitro, cyano, carbamoyl, thiocarbamoyl, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, trichloroethyl, chlorodifluoroethyl, fluorodichloroethyl, fluoropropyl, fluoro-i-propyl, chloropropyl, chloro-i-propyl, methoxy, ethoxy, n- or i-propoxy, n-, i-, s- or t-butoxy, fluoromethoxy, difluoro-methoxy, trifluoromethoxy, chlorodifluoromethoxy, fluoroethoxy, chloroethoxy, difluoro-ethoxy, dichloroethoxy, trifluoroethoxy, tetrafluoroethoxy, pentafluoroethoxy, fluoro-propoxy, chloropropoxy, difluoropropoxy, trifluoropropoxy, tetrafluoropropoxy, methoxy-methyl, ethoxymethyl, propoxymethyl, i-propoxymethyl, methoxyethyl, ethoxyethyl, methylthio, ethylthio, n- or i-propylthio, n-, i-, s- or t-butylthio, difluoromethylthio, trifluoro-methylthio, chlorodifluoromethylthio, fluoroethylthio, chloroethylthio, difluoroethylthio, dichloroethylthio, trifluoroethylthio, methylsulfinyl, ethylsulfinyl, propylsulfinyl, difluoro-methylsulfinyl, trifluoromethylsulfinyl, methylsulfonyl, ethylsulfonyl, trifluoromethyl-sulfonyl, phenyl, phenoxy, phenylthio, benzyl, phenylethyl, phenylmethoxy, phenylethoxy, phenylmethylthio, phenylethylthio (where in each case the phenyl groups of the substituents optionally contain one or more of the substituents listed above, in particular cyano, fluorine, chlorine, bromine, methyl, trifluoromethyl or trifluoromethoxy), heterocyclyl, heterocyclyloxy, heterocyclylthio, heterocyclylmethyl, heterocyclylmethoxy, heterocyclyl-methylthio, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl.
Ar especially preferably represents mono- to trisubstituted phenyl, the following substituents being possible:
   nitro, cyano, fluorine, chlorine, bromine, methyl, trifluoromethyl, trifluoromethoxy or phenyl (which contains one or more of the substituents listed above),
Ar furthermore especially preferably represents methoxy- or iodine-substituted phenyl.
R preferably represents hydrogen or one of the groupings below

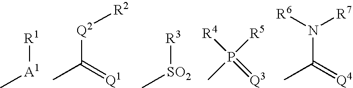

where
A¹ represents a carbonyl group (C═O) or represents methylene,
Q¹, Q², Q³ and Q⁴ each represent O (oxygen) or S (sulfur),
R¹ represents optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted alkyl having 1 to 10 carbon atoms, represents in each case optionally cyano-, halogen- or C₁-C₆-alkoxy-carbonyl-substituted alkenyl or alkynyl having in each case 2 to 10 carbon atoms, represents in each case optionally cyano-, halogen-, C₁-C₆-alkyl- or C₁-C₆-haloalkyl-substituted cycloalkyl or cycloalkylalkyl having in each case 3 to 6 carbon atoms in the cycloalkyl group and optionally 1 to 4 carbon atoms in the alkyl moiety, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclylalkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 4 carbon atoms, and, if A¹ is methylene, (R¹) also represents in each case optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted alkoxy, alkylthio, alkylsulfinyl, alkylsulfonyl, alkylamino, dialkylamino, alkyl-carbonylamino, N-alkyl-alkylcarbonylamino, alkoxycarbonylamino or N-alkyl-alkoxycarbonylamino having in each case 1 to 10 carbon atoms in the alkyl groups, represents in each case optionally cyano-, halogen- or C₁-C₆-alkoxy-carbonyl-substituted alkenyloxy, alkynyloxy, alkenylamino, alkynylamino, N—(C₁-C₆-alkyl)-alkenylamino or N—(C₁-C₆-alkyl)-alkynylamino having in each case 2 to 10 carbon atoms in the alkenyl or alkynyl groups, represents in each case optionally cyano-, halogen-, C₁-C₆-alkyl- or C₁-C₆-haloalkyl-substituted cycloalkyloxy, cycloalkylalkoxy, cycloalkylamino, cycloalkylalkylamino, N—(C₁-C₆-alkyl)-cycloalkylamino or N—(C₁-C₆-alkyl)-cycloalkylalkylamino having in each case 3 to 6 carbon atoms in the cycloalkyl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryloxy, arylalkoxy, arylamino, arylalkylamino, N-alkyl-arylamino or N-alkyl-arylalkylamino having in each case 6 or 10 carbon atoms in the aryl group and, if appropriate, 1 to 4 carbon atoms in the alkyl moiety,
R² represents optionally cyano-, halogen- or C₁-C₆-alkoxy-substituted alkyl having 1 to 6 carbon atoms, represents in each case optionally cyano- or halogen-substituted alkenyl or alkynyl having in each case 3 to 6 carbon atoms, represents in each case optionally cyano-, halogen- or C₁-C₆-alkyl-substituted cycloalkyl or cycloalkylalkyl having in each case 3 to 6 carbon atoms in the cycloalkyl group and, if appropriate, 1 to 4 carbon atoms in the alkyl moiety, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclylalkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 4 carbon atoms,
R³ represents optionally halogen-substituted alkyl having 1 to 10 carbon atoms, represents in each case optionally halogen-substituted alkenyl or alkynyl having in each case 2 to 10 carbon atoms, represents in each case optionally halogen-, C₁-C₆-alkyl- or C₁-C₆-haloalkyl-substituted cycloalkyl or cycloalkylalkyl having in each case 3 to 6 carbon atoms in the cycloalkyl group and, if appropriate, 1 to 4 carbon atoms in the alkyl moiety, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclylalkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 4 carbon atoms,
R⁴ and R⁵ are identical or different and independently of one another represent in each case optionally cyano-, halogen- or C₁-C₆-alkoxy-substituted alkyl, alkoxy, alkylthio, alkylamino or dialkylamino having in each case 1 to 6 carbon atoms in the alkyl groups, or represent in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl, phenoxy or phenylthio,
R⁶ represents hydrogen, represents optionally cyano-, halogen- or C₁-C₆-alkoxy-substituted alkyl having 1 to 6 carbon atoms or represents optionally cyano- or halogen-substituted alkenyl having 2 to 6 carbon atoms, and
R⁷ represents hydrogen, represents in each case optionally cyano-, halogen- or C₁-C₆-alkoxy-substituted alkyl or alkoxy having in each case 1 to 6 carbon atoms, represents optionally cyano- or halogen-substituted alkenyl having 2 to 6 carbon atoms, represents in each case optionally cyano-, halogen- or C₁-C₆-alkyl-substituted cycloalkyl or cycloalkylalkyl having in each case 3 to 6 carbon atoms in the cycloalkyl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or together with R⁶ represents optionally hydroxyl-, cyano- or C₁-C₆-alkyl-substituted C₂-C₆-alkanediyl in which optionally one methylene grouping is replaced by O (oxygen) or S (sulfur).
R particularly preferably represents hydrogen or one of the groupings below

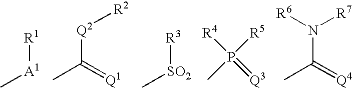

where
A¹ represents a carbonyl group (C═O) or represents methylene,
Q¹, Q², Q³ and Q⁴ each represent O (oxygen) or S (sulfur),
R¹ represents a optionally cyano-, halogen-, C₁-C₅-alkoxy-, C₁-C₅-alkylthio-, C₁-C₅-alkyl-sulfinyl- or C₁-C₅-alkylsulfonyl-substituted alkyl having 1 to 6 carbon atoms, represents in each case optionally cyano- or halogen-substituted alkenyl or alkynyl having in each case 2 to 6 carbon atoms, represents in each case optionally cyano-, halogen-, C₁-C₅-alkyl- or C₁-C₅-haloalkyl-substituted cycloalkyl or cycloalkylalkyl having in each case 3 to 6 carbon atoms in the cycloalkyl group and, if appropriate, 1 to 4 carbon atoms in the alkyl moiety, represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted aryl or arylalkyl having in each case 6 or 10 carbon atoms in the aryl group and, if appropriate, 1 to 3 carbon atoms in the alkyl moiety, or represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted heterocyclyl or heterocyclylalkyl, where the heterocyclyl grouping in each case contains 1 to 5 carbon atoms and 1 to 3 atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 3 carbon atoms, and, if A¹ represents methylene, (R¹) also represents in each case optionally cyano-, halogen-, C₁-C₅-alkoxy-, C₁-C₅-alkylthio-, C₁-C₅-alkylsulfinyl-, C₁-C₅-alkylsulfonyl-, C₁-C₅-alkylamino- or di(C₁-C₅-alkyl)-amino-substituted alkoxy, alkylthio, alkylsulfinyl, alkylsulfonyl, alkylamino, dialkylamino, alkylcarbonylamino, N-alkyl-alkylcarbonylamino, alkoxycarbonylamino or N-alkyl-alkoxycarbonylamino having in each case 1 to 6 carbon atoms in the alkyl groups, represents in each case optionally cyano-, halogen- or C₁-C₅-alkoxy-carbonyl-substituted alkenyloxy, alkynyloxy, alkenylamino, alkynylamino, N—(C₁-C₅-alkyl)-alkenylamino or N—(C₁-C₅-alkyl)-alkynylamino having in each case 2 to 6 carbon atoms in the alkenyl or alkynyl groups, represents in each case optionally cyano-, halogen-, C₁-C₅-alkyl- or C₁-C₅-haloalkyl-substituted cycloalkyloxy, cycloalkylalkoxy, cycloalkylamino, cycloalkylalkylamino, N—(C₁-C₅-alkyl)-cycloalkylamino or N—(C₁-C₅-alkyl)-cycloalkylalkylamino having in each case 3 to 6 carbon atoms in the cycloalkyl group and, if appropriate, 1 to 3 carbon atoms in the alkyl moiety, or represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₃-alkylenedioxy- or C₁-C₃-haloalkylenedioxy-substituted aryloxy, arylalkoxy, arylamino, arylalkylamino, N-alkyl-arylamino or N-alkyl-arylalkylamino having in each case 6 or 10 carbon atoms in the aryl group and, if appropriate, 1 to 3 carbon atoms in the alkyl moiety,
R² represents optionally cyano-, halogen- or C₁-C₅-alkoxy-substituted alkyl having 1 to 5 carbon atoms, represents in each case optionally cyano- or halogen-substituted alkenyl or alkynyl having in each case 3 to 5 carbon atoms, represents in each case optionally cyano-, halogen- or C₁-C₅-alkyl-substituted cycloalkyl or cycloalkylalkyl having in each case 3 to 6 carbon atoms in the cycloalkyl group and, if appropriate, 1 to 3 carbon atoms in the alkyl moiety, represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted aryl or arylalkyl having in each case 6 or 10 carbon atoms in the aryl group and, if appropriate, 1 to 3 carbon atoms in the alkyl moiety, or represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted heterocyclyl or heterocyclylalkyl, where the heterocyclyl grouping in each case contains 1 to 5 carbon atoms and 1 to 3 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 3 carbon atoms,
R³ represents optionally halogen-substituted alkyl having 1 to 6 carbon atoms, represents in each case optionally halogen-substituted alkenyl or alkynyl having in each case 2 to 6 carbon atoms, represents in each case optionally halogen-, C₁-C₅-alkyl- or C₁-C₅-haloalkyl-substituted cycloalkyl or cycloalkylalkyl having in each case 3 to 6 carbon atoms in the cycloalkyl group and, if appropriate, 1 to 3 carbon atoms in the alkyl moiety, represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted aryl or arylalkyl having in each case 6 or 10 carbon atoms in the aryl group and, if appropriate, 1 to 3 carbon atoms in the alkyl moiety, or represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted heterocyclyl or heterocyclylalkyl, where the heterocyclyl grouping in each case contains 1 to 6 carbon atoms and 1 to 3 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 3 carbon atoms,
R⁴ and R⁵ are identical or different and independently of one another represent in each case optionally cyano-, halogen- or C₁-C₆-alkoxy-substituted alkyl, alkoxy, alkylthio, alkylamino or dialkylamino having in each case 1 to 5 carbon atoms in the alkyl groups, or represent in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkyl-thio-, C₁-C₅-alkoxy-carbonyl-, C₁-C₂-alkylenedioxy- or C₁-C₂-haloalkylenedioxy-substituted phenyl, phenoxy or phenylthio,
R⁶ represents hydrogen, represents optionally cyano-, halogen- or C₁-C₅-alkoxy-substituted alkyl having 1 to 5 carbon atoms or represents optionally cyano- or halogen-substituted alkenyl having 2 to 5 carbon atoms, and
R⁷ represents hydrogen, represents in each case optionally cyano-, halogen- or C₁-C₅-alkoxy-substituted alkyl or alkoxy having in each case 1 to 5 carbon atoms, represents optionally cyano- or halogen-substituted alkenyl having 2 to 5 carbon atoms, represents in each case optionally cyano-, halogen- or C₁-C₅-alkyl-substituted cycloalkyl or cycloalkylalkyl having in each case 3 to 6 carbon atoms in the cycloalkyl group and, if appropriate, 1 to 3 carbon atoms in the alkyl moiety, or represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl-, C₁-C₂-alkylenedioxy- or C₁-C₂-haloalkylenedioxy-substituted aryl or arylalkyl having in each case 6 or 10 carbon atoms in the aryl group and, if appropriate, 1 to 4 carbon atoms in the alkyl moiety, or together with R⁶ represent optionally hydroxyl-, cyano- or C₁-C₅-alkyl-substituted C₂-C₅-alkanediyl in which optionally one methylene grouping is replaced by O (oxygen) or S (sulfur).
R very particularly preferably represents hydrogen or one of the groupings below

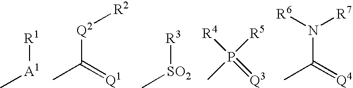

where
A¹ represents a carbonyl group (C═O) or represents methylene,
Q¹, Q², Q³ and Q⁴ each represent O (oxygen) or S (sulfur),
R¹ represents in each case optionally cyano-, fluorine-, chlorine-, bromine-, methoxy-, ethoxy-, n- or i-propoxy-, n-, i-, s- or t-butoxy-, methylthio-, ethylthio-, n- or i-propyl-thio-, n-, i-, s- or t-butylthio-, methylsulfinyl-, ethylsulfinyl-, propylsulfinyl-, methyl-sulfonyl- or ethylsulfonyl-substituted methyl, ethyl, n- or i-propyl, n-, i-, s- or t-butyl, n-, i-, s-, t- or neo-pentyl, represents in each case optionally cyano-, fluorine-, chlorine- or bromine-substituted ethenyl, propenyl, butenyl, pentenyl, ethynyl, propynyl, butynyl or pentynyl, represents in each case optionally cyano-, fluorine-, chlorine-, bromine-, methyl-, ethyl-, n- or i-propyl- or trifluoromethyl-substituted cyclopropyl, cyclobutyl, cyclopentyl, cyclohexyl, cyclopropylmethyl, cyclobutylmethyl, cyclopentylmethyl or cyclohexylmethyl, represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluorodichloroethyl-, fluoropropyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, trifluoroethoxy-, trichloroethoxy-, chlorodifluoroethoxy-, fluorodichloroethoxy-, tetrafluoroethoxy-, chlorotrifluoroethoxy-, pentafluoroethoxy-, methylthio-, ethylthio-, n- or i-propylthio-, n-, i-, s- or t-butylthio-, difluoromethylthio-, trifluoromethylthio-, chlorodifluoromethylthio-, fluoroethylthio-, chloroethylthio-, difluoroethylthio-, dichloroethylthio-, chlorofluoroethylthio-, trifluoroethylthio-, chlorodifluoroethylthio-, methoxycarbonyl-, ethoxycarbonyl-, n- or i-propoxycarbonyl-, n-, i-, s- or t-butoxy-carbonyl-, methylenedioxy-, dimethylenedioxy-, difluoromethylenedioxy-, difluorodimethylenedioxy-, trifluorodimethylenedioxy- or chlorodifluorodimethylenedioxy-substituted phenyl, naphthyl, benzyl, phenylethyl, or represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluorodichloroethyl-, fluoropropyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, trifluoroethoxy-, trichloroethoxy-chlorodifluoroethoxy-, fluorodichloroethoxy-, tetrafluoroethoxy-, chlorotrifluoro-ethoxy-, pentafluoroethoxy-, methylthio-, ethylthio-, n- or i-propylthio-, n-, i-, s- or t-butylthio-, difluoromethylthio-, trifluoromethylthio-, chlorodifluoromethylthio-, fluoroethylthio-, chloroethylthio-, difluoroethylthio-, dichloroethylthio-, chlorofluoro-ethylthio-, trifluoroethylthio-, chlorodifluoroethylthio-, methoxycarbonyl-, ethoxy-carbonyl-, n- or i-propoxycarbonyl-, n-, i-, s- or t-butoxycarbonyl-, methylenedioxy-, dimethylenedioxy-, difluoromethylenedioxy-, difluorodimethylenedioxy-, trifluoro-methylenedioxy- or chlorodifluorodimethylenedioxy-substituted heterocyclyl or heterocyclylmethyl, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl, and, if A¹ represents methylene, (R¹) also represents in each case optionally cyano-, fluorine-, chlorine-, bromine-, methoxy-, ethoxy-, n- or i-propoxy-, n-, i-, s- or t-butoxy-, methylthio-, ethylthio-, n- or i-propylthio-, n-, i-, s- or t-butylthio-, methylsulfinyl-, ethylsulfinyl-, propylsulfinyl-, methylsulfonyl-, ethylsulfonyl-, methylamino-, ethylamino-, n- or i-propylamino-, n-, i-, s- or t-butylamino-, dimethylamino-, diethylamino- or dipropyl-amino-substituted methoxy, ethoxy, n- or i-propoxy, n-, i-, s- or t-butoxy, n-, i-, s- or t-pentoxy, methylthio, ethylthio, n- or i-propylthio, n-, i-, s- or t-butylthio, methyl-sulfinyl, ethylsulfinyl, propylsulfinyl, methylsulfonyl, ethylsulfonyl, methylamino, ethylamino, n- or i-propylamino, n-, i-, s- or t-butylamino, dimethylamino, diethylamino, dipropylamino, acetylamino, propionylamino, butyrylamino, N-methyl-acetylamino, N-ethyl-acetylamino, N-methyl-propionylamino, N-ethyl-propionylamino, methoxycarbonylamino, ethoxycarbonylamino, n- or i-propoxycarbonylamino, N-methyl-methoxycarbonylamino, N-ethyl-methoxycarbonylamino, N-methyl-ethoxy-carbonylamino or N-ethyl-ethoxycarbonylamino, represents in each case optionally cyano-, fluorine-, chlorine-, bromine-, methoxycarbonyl-, ethoxycarbonyl-, n- or i-propoxycarbonyl-substituted propenyloxy, butenyloxy, pentenyloxy, propynyloxy, butynyloxy, pentynyloxy, propenylamino, butenylamino, pentenylamino, propynyl-amino, butynylamino, pentynylamino, N-methyl-propenylamino, N-ethyl-propenyl-amino, N-methyl-butenylamino, N-ethyl-butenylamino, N-methyl-propynylamino, N-ethyl-propynylamino, N-methyl-butynylamino or N-ethyl-butynylamino, represents in each case optionally cyano-, fluorine-, chlorine-, bromine-, methyl-, ethyl-, n- or i-propyl- or trifluoromethyl-substituted cyclopropyloxy, cyclobutyloxy, cyclo-pentyloxy, cyclohexyloxy, cyclopropylmethoxy, cyclobutylmethoxy, cyclopentyl-methoxy, cyclohexylmethoxy, cyclopropylamino, cyclobutylamino, cyclopentylamino, cyclohexylamino, cyclopropylmethylamino, cyclobutylmethylamino, cyclopentyl-methylamino, cyclohexylmethylamino, N-methyl-cyclopropylamino, N-methyl-cyclo-butylamino, N-methyl-cyclopentylamino, N-methyl-cyclohexylamino, N-methyl-cyclo-propylmethylamino, N-methyl-cyclobutylmethylamino, N-methyl-cyclopentylmethyl-amino or N-methyl-cyclohexylmethylamino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, chlorodifluoromethoxy-, methylthio-, ethylthio-, n- or i-propylthio-, n-, i-, s- or t-butylthio-, difluoromethylthio-, trifluoromethylthio-, chlorodifluoromethylthio-, methoxycarbonyl-, ethoxycarbonyl-, n- or i-propoxycarbonyl-, methylenedioxy-, dimethylenedioxy-, difluoromethylenedioxy-, difluorodimethylenedioxy-, trifluorodimethylenedioxy- or tetrafluorodimethylene-dioxy-substituted phenyloxy, phenylmethoxy, phenylethoxy, phenylamino, phenyl-methylamino, phenyl ethyl amino, N-methyl-phenylamino, N-ethyl-phenylamino, N-methyl-phenylmethylamino, N-ethyl-phenylmethylamino, N-methyl-phenylethyl-amino or N-ethyl-phenylethylamino,
R¹ furthermore represents in each case optionally cyano-, fluorine-, chlorine-, bromine-, methoxy-, ethoxy-, n- or i-propoxy-, n-, i-, s- or t-butoxy-, methylthio-, ethylthio-, n- or i-propylthio-, n-, i-, s- or t-butylthio-, methylsulfinyl-, ethylsulfinyl-, propylsulfinyl-, methylsulfonyl- or ethylsulfonyl-substituted 1,1-dimethylpropyl, 1,2-dimethylpropyl or 1,1,2-trimethylpropyl,
R² represents in each case optionally cyano-, fluorine-, chlorine-, methoxy-, ethoxy-, n- or i-propoxy-, n-, i-, s- or t-butoxy-substituted methyl, ethyl, n- or i-propyl, n-, i-, s- or t-butyl, represents in each case optionally cyano-, fluorine-, chlorine- or bromine-substituted propenyl, butenyl, propynyl or butynyl, represents in each case optionally cyano-, fluorine-, chlorine-, bromine-, methyl-, ethyl-, n- or i-propyl-substituted cyclopropyl, cyclobutyl, cyclopentyl, cyclohexyl, cyclopropylmethyl, cyclobutyl-methyl, cyclopentylmethyl or cyclohexylmethyl, represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloro-methyl-, fluoroethyl-, chloroethyl-, difluoroethyl-, dichloroethyl, chlorofluoroethyl-, trifluoroethyl-, trichloroethyl-, chlorodifluoroethyl-, fluorodichloroethyl-, fluoro-propyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoro-ethoxy-, chloroethoxy-, difluoroethoxy-, dichloroethoxy-, chlorofluoroethoxy-, trifluoroethoxy-, trichloroethoxy-, chlorodifluoroethoxy-, fluorodichloroethoxy-, tetrafluoroethoxy-, chlorotrifluoroethoxy-, pentafluoroethoxy-, methylthio-, ethylthio-, n- or i-propylthio-, n-, i-, s- or t-butylthio-, difluoromethylthio-, trifluoromethylthio-, chlorodifluoromethylthio-, fluoroethylthio-, chloroethylthio-, difluoroethylthio-, dichloroethylthio-, chlorofluoroethylthio-, trifluoroethylthio, chlorodifluoroethylthio-, methoxycarbonyl-, ethoxycarbonyl-, n- or i-propoxycarbonyl-, n-, i-, s- or t-butoxy-carbonyl-, methylenedioxy-, dimethylenedioxy-, difluoromethylenedioxy-, difluoro-dimethylenedioxy-, trifluorodimethylenedioxy- or chlorodifluorodimethylenedioxy-substituted phenyl, naphthyl, benzyl or phenylethyl, or represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl, tri-chloromethyl-, fluoroethyl-, chloroethyl-, difluoroethyl-, dichloroethyl-, chlorofluoro-ethyl-, trifluoroethyl-, trichloroethyl-, chlorodifluoroethyl-, fluorodichloroethyl-, fluoropropyl-, fluoro-i-propyl-, methoxy-, ethoxy-, n- or i-propoxy-, n-, i, s- or t-butoxy-, fluoromethoxy-, difluoromethoxy-, trifluoromethoxy-, chlorodifluoro-methoxy-, fluoroethoxy-, chloroethoxy-, difluoroethoxy-, dichloroethoxy-, chlorofluoroethoxy-, trifluoroethoxy-, trichloroethoxy-, chlorodifluoroethoxy-, fluorodichloroethoxy-, tetrafluoroethoxy-, chlorotrifluoroethoxy-, pentafluoroethoxy-, methylthio-, ethylthio-, n- or i-propylthio-, n-, i-, s- or t-butylthio-, difluoromethylthio-, trifluoromethylthio-, chlorodifluoromethylthio-, fluoroethylthio-, chloroethylthio-, difluoroethylthio-, dichloroethylthio-, chlorofluoroethylthio-, trifluoroethylthio-, chlorodifluoroethylthio-, methoxycarbonyl-, ethoxycarbonyl-, n- or i-propoxy-carbonyl-, n-, i-, s- or t-butoxycarbonyl-, methylenedioxy-, dimethylenedioxy-, difluoromethylenedioxy-, difluorodimethylenedioxy-, trifluorodimethylenedioxy- or chlorodifluorodimethylenedioxy-substituted heterocyclyl or heterocyclylmethyl, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl.
R³ represents in each case optionally fluorine-, chlorine- or bromine-substituted methyl, ethyl, n- or i-propyl, n-, i-, s- or t-butyl, represents in each case optionally fluorine-, chlorine- or bromine-substituted ethenyl, propenyl, butenyl, ethynyl, propynyl or butynyl, represents in each case optionally fluorine-, chlorine-, bromine-, methyl-, ethyl-, n- or i-propyl- or trifluoromethyl-substituted cyclopropyl, cyclobutyl, cyclo-pentyl, cyclohexyl, cyclopropylmethyl, cyclobutylmethyl, cyclopentylmethyl or cyclohexylmethyl, represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloromethyl-, fluoroethyl-, chloroethyl-, difluoroethyl-, dichloroethyl-, chlorofluoroethyl-, trifluoroethyl-, tri-chloroethyl-, chlorodifluoroethyl-, fluorodichloroethyl-, fluoropropyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, chloro-ethoxy-, difluoroethoxy-, dichloroethoxy-, chlorofluoroethoxy-, trifluoroethoxy-, trichloroethoxy-, chlorodifluoroethoxy-, fluorodichloroethoxy-, tetrafluoroethoxy-, chlorotrifluoroethoxy-, pentafluoroethoxy-, methylthio-, ethylthio-, n- or i-propylthio-, n-, i-, s- or t-butylthio-, difluoromethylthio-, trifluoromethylthio-, chlorodifluoro-methylthio-, fluoroethylthio-, chloroethylthio-, difluoroethylthio-, dichloroethylthio-, chlorofluoroethylthio-, trifluoroethylthio-, chlorodifluoroethylthio-, methoxycarbonyl-, ethoxycarbonyl-, n- or i-propoxycarbonyl-, n-, i-, s- or t-butoxycarbonyl-, methylene-dioxy-, dimethylenedioxy-, difluoromethylenedioxy-, difluorodimethylenedioxy-, trifluorodimethylenedioxy- or chlorodifluorodimethylenedioxy-substituted phenyl, naphthyl, benzyl or phenylethyl, or represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloromethyl-, fluoro-ethyl-, chloroethyl-, difluoroethyl-, dichloroethyl-, chlorofluoroethyl-, trifluoroethyl-, trichloroethyl-, chlorodifluoroethyl-, fluorodichloroethyl-, fluoropropyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, chloro-ethoxy-, difluoroethoxy-, dichloroethoxy-, chlorofluoroethoxy-, trifluoroethoxy-, trichloroethoxy-, chlorodifluoroethoxy-, fluorodichloroethoxy-, tetrafluoroethoxy-, chlorotrifluoroethoxy-, pentafluoroethoxy-, methylthio-, ethylthio-, n- or i-propylthio-, n-, i-, s- or t-butylthio-, difluoromethylthio-, trifluoromethylthio-, chlorodifluoro-methylthio-, fluoroethylthio-, chloroethylthio-, difluoroethylthio-, dichloroethylthio-, chlorofluoroethylthio-, trifluoroethylthio-, chlorodifluoroethylthio-, methoxycarbonyl-, ethoxycarbonyl-, n- or i-propoxycarbonyl-, n-, i-, s- or t-butoxycarbonyl-, methylenedioxy-, dimethylenedioxy-, difluoromethylenedioxy-, difluorodimethylene-dioxy-, trifluorodimethylenedioxy- or chlorodifluorodimethylenedioxy-substituted heterocyclyl or heterocyclylmethyl, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl.
R⁴ and R⁵ are identical or different and independently of one another represent in each case optionally cyano-, fluorine-, chlorine-, bromine-, methoxy-, ethoxy-, n- or i-propoxy-, n-, i-, s- or t-butoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino, or represent in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluorodichloroethyl-, fluoropropyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoro-methoxy-, fluoroethoxy-, chloroethoxy-, difluoroethoxy-, dichloroethoxy-, chlorofluoroethoxy-, trifluoroethoxy-, trichloroethoxy-, chlorodifluoroethoxy-, fluoro-dichloroethoxy-, tetrafluoroethoxy-, chlorotrifluoroethoxy-, pentafluoroethoxy-, methylthio-, ethylthio-, n- or i-propylthio-, n-, i-, s- or t-butylthio-, difluoromethylthio-, trifluoromethylthio-, chlorodifluoromethylthio-, fluoroethylthio-, chloroethylthio-, difluoroethylthio-, dichloroethylthio-, chlorofluoroethylthio-, trifluoroethylthio-, chlorodifluoroethylthio-, methoxycarbonyl-, ethoxycarbonyl-, n- or i-propoxy-carbonyl-, n-, i-, s- or t-butoxycarbonyl-, methylenedioxy-, dimethylenedioxy-, difluoromethylenedioxy-, difluorodimethylenedioxy-, trifluorodimethylenedioxy- or chlorodifluorodimethylenedioxy-substituted phenyl, phenoxy or phenylthio,
R⁶ represents hydrogen, represents in each case optionally cyano-, fluorine-, chlorine-, methoxy-, ethoxy-, n- or i-propoxy-, n-, i-, s- or t-butoxy-substituted methyl, ethyl, n- or i-propyl, n-, i-, s- or t-butyl, or represents in each case optionally cyano-, fluorine-, chlorine- or bromine-substituted propenyl or butenyl, and
R⁷ represents hydrogen, represents in each case optionally cyano-, fluorine-, chlorine-, methoxy-, ethoxy-, n- or i-propoxy-, n-, i-, s- or t-butoxy-substituted methyl, ethyl, n- or i-propyl, n-, i-, s- or t-butyl, methoxy, ethoxy, n- or i-propoxy, n-, i-, s- or t-butoxy, represents in each case optionally cyano-, fluorine-, chlorine- or bromine-substituted propenyl or butenyl, represents in each case optionally cyano-, fluorine-, chlorine-, methyl-, ethyl-, n- or i-propyl-substituted cyclopropyl, cyclobutyl, cyclopentyl, cyclohexyl, cyclopropylmethyl, cyclobutylmethyl, cyclopentylmethyl or cyclo-hexylmethyl, or represents in each case optionally nitro-, cyano-, fluorine-, chlorine-, bromine-, iodine-, methyl-, ethyl-, n- or i-propyl-, n-, i-, s- or t-butyl-, difluoromethyl-, trifluoromethyl-, dichloromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluorodichloroethyl-, fluoropropyl-, fluoro-i-propyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, chloroethoxy-, difluoro-ethoxy-, dichloroethoxy-, chlorofluoroethoxy-, trifluoroethoxy-, trichloroethoxy-, chlorodifluoroethoxy-, fluorodichloroethoxy-, tetrafluoroethoxy-, chlorotrifluoro-ethoxy-, pentafluoroethoxy-, methylthio-, ethylthio-, n- or i-propylthio-, n-, i-, s- or t-butylthio-, difluoromethylthio-, trifluoromethylthio-, chlorodifluoromethylthio-, fluoroethylthio-, chloroethylthio-, difluoroethylthio-, dichloroethylthio-, chlorofluoroethylthio-, trifluoroethylthio-, chlorodifluoroethylthio-, methoxycarbonyl-, ethoxycarbonyl-, n- or i-propoxycarbonyl-, n-, i-, s- or t-butoxycarbonyl-, methylenedioxy-, dimethylenedioxy-, difluoromethylenedioxy-, difluorodimethylene-dioxy-, trifluorodimethylenedioxy- or chlorodifluorodimethylenedioxy-substituted phenyl, naphthyl, benzyl or phenyl ethyl, or together with R⁶ represents optionally hydroxyl-, cyano-, methyl-, ethyl-, n- or i-propyl-substituted ethane-1,2-diyl (dimethylene), propane-1,3-diyl (trimethylene), butane-1,4-diyl (tetramethylene) or pentane-1-5-diyl (pentamethylene), where in each case one methylene grouping is optionally replaced by O (oxygen) or S (sulfur).
R especially preferably represents hydrogen or one of the groupings below

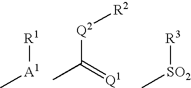

where
A¹ represents a carbonyl group (C═O) or represents methylene,
Q¹, Q² each represent 0,
R¹ represents methyl, t-butyl or represents optionally chlorine-substituted phenyl,
R¹ furthermore represents 1,1,2-trimethylpropyl or ethoxy,
R² represents i-propyl,
R³ represents optionally methyl-substituted phenyl.

According to the invention, preference is given to the use according to the invention of compounds of the formula (I) which contain a combination of the meanings given above as being preferred.

According to the invention, particular preference is given to the use according to the invention of compounds of the formula (I) which contain a combination of the meanings given above as being particularly preferred.

According to the invention, very particular preference is given to the use according to the invention of compounds of the formula (I) which contain a combination of the meanings given above as being very particularly preferred.

A small number of the compounds of the general formula (I) to be used according to the invention is already known (cf. the literature cited at the outset).

A large number of the compounds of the general formula (I) to be used according to the invention is not yet known from the literature.

Therefore, the compounds of the general formula (IA)—below—are also claimed as novel compounds according to the invention.

A particular group of the compounds to be used according to the invention which, as novel compounds, form part of the subject-matter of the present application, is also represented by the formula (IA)

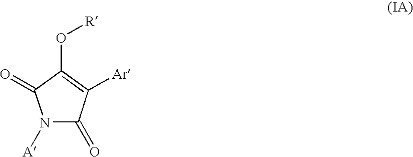

in which
A′ represents optionally cyano-, halogen-, C₁-C₆-alkoxy-, C₁-C₆-alkylthio-, C₁-C₆-alkylsulfinyl-, C₁-C₆-alkylsulfonyl- or C₁-C₆-alkoxy-carbonyl-substituted C₁-C₈-alkyl, represents in each case optionally cyano- or halogen-substituted C₂-C₈-alkenyl or C₂-C₈-alkynyl, represents in each case optionally cyano-, halogen-, C₁-C₆-alkyl-, C₁-C₆-haloalkyl-, C₁-C₆-alkoxy- or C₁-C₆-haloalkoxy-substituted C₃-C₅-cycloalkyl, C₇-C₈-cycloalkyl, C₅-C₆-cycloalkenyl or C₃-C₈-cycloalkyl-C₁-C₄-alkyl, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-halo-alkylthio-, C₁-C₆-alkoxy-carbonyl- or C₁-C₃-alkylenedioxy-substituted phenyl or phenyl-C₁-C₄-alkyl, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl- or C₁-C₃-alkylenedioxy-substituted heterocyclyl or heterocyclyl-C₁-C₄-alkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping, or (A′) represents the cyclohexyl grouping below

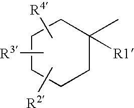

in which
R^{1′} represents hydrogen, cyano, C₁-C₆-alkyl or C₁-C₆-alkoxy-carbonyl,
R^{2′}, R^{3′} and R^{4′} are identical or different and individually represent hydrogen, halogen, C₁-C₆-alkyl, C₁-C₆-haloalkyl, C₁-C₆-alkoxy, C₁-C₆-haloalkoxy or benzyloxy, or two of these radicals (if adjacent) together represent C₃-C₅-alkanediyl which is optionally interrupted by 1 or 2 heteroatoms from the group consisting of N, O, S.
A′ furthermore represents optionally cyano-, halogen-, C₁-C₆-alkyl, C₁-C₆-haloalkyl-, C₁-C₆-alkoxy- or C₁-C₆-haloalkoxy-substituted adamantyl, or (A′) represents the cyclohexyl grouping below

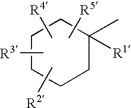

in which
R^{1′} represents hydrogen, cyano, C₁-C₆-alkyl or C₁-C₆-alkoxy-carbonyl,
R^{2′}, R^{3′}, R^{4′} and R^{5′} are identical or different and individually represent hydrogen, halogen, C₁-C₆-alkyl, C₁-C₆-haloalkyl, C₁-C₆-alkoxy, C₁-C₆-haloalkoxy, C₁-C₆-alkoxy-carbonyl or benzyloxy, or two of these radicals (if adjacent) together represent C₃-C₅-alkanediyl which is optionally interrupted by 1 or 2 heteroatoms from the group consisting of N, O, S.
Ar′ represents phenyl which is mono- to pentasubstituted by identical or different substituents, except for 3,4-dimethoxyphenyl and 3,4-methylenedioxyphenyl, which are excluded by disclaimer, suitable substituents being—with the above disclaimer being taken into account—the following substituents:
   nitro, cyano, carbamoyl, thiocarbamoyl, halogen, C₁-C₆-alkyl, C₁-C₆-haloalkyl, C₂-C₆-alkoxy, C₁-C₆-haloalkoxy, C₁-C₄-alkoxy-C₁-C₄-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, phenyl (which contains one or more of the substituents listed above), phenoxy (which optionally contains one or more of the substituents listed above), phenylthio (which optionally contains one or more of the substituents listed above), phenyl-C₁-C₄-alkyl (which optionally contains one or more of the substituents listed above), phenyl-C₁-C₄-alkoxy (which optionally contains one or more of the substituents listed above), phenyl-C₁-C₄-alkylthio (which optionally contains one or more of the substituents listed above), heterocyclyl (which optionally contains one or more of the substituents listed above), heterocyclyloxy (which optionally contains one or more of the substituents listed above), heterocyclylthio (which optionally contains one or more of the substituents listed above), heterocyclyl-C₁-C₄-alkyl (which optionally contains one or more of the substituents listed above), heterocyclyl-C₁-C₄-alkoxy (which optionally contains one or more of the substituents listed above), heterocyclyl-C₁-C₄-alkylthio (which optionally contains one or more of the substituents listed above), where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping, and
R′ represents hydrogen or one of the groupings below

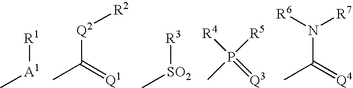

where
A¹ represents a carbonyl group (C═O) or represents methylene,
Q¹, Q², Q³ and Q⁴ each represent O (oxygen) or S (sulfur),
R¹ represents optionally cyano-, halogen-, C₁-C₆-alkoxy-, C₁-C₆-alkylthio-, C₁-C₆-alkylsulfinyl- or C₁-C₆-alkylsulfonyl-substituted alkyl having 1 to 10 carbon atoms, represents in each case optionally cyano- or halogen-substituted alkenyl or alkynyl having in each case 2 to 10 carbon atoms, represents in each case optionally cyano-, halogen-, C₁-C₆-alkyl- or C₁-C₆-haloalkyl-substituted cycloalkyl or cycloalkylalkyl having in each case 3 to 6 carbon atoms in the cycloalkyl group and, if appropriate, 1 to 6 carbon atoms in the alkyl moiety, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or having in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclylalkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 4 carbon atoms,
R² represents optionally cyano-, halogen- or C₁-C₆-alkoxy-substituted alkyl having 1 to 6 carbon atoms, represents in each case optionally cyano- or halogen-substituted alkenyl or alkynyl having in each case 3 to 6 carbon atoms, represents in each case optionally cyano-, halogen- or C₁-C₆-alkyl-substituted cycloalkyl or cycloalkylalkyl having in each case 3 to 6 carbon atoms in the cycloalkyl group and, if appropriate, 1 to 4 carbon atoms in the alkyl moiety, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclylalkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 4 carbon atoms,
R³ represents optionally halogen-substituted alkyl having 1 to 10 carbon atoms, represents in each case optionally halogen-substituted alkenyl or alkynyl having in each case 2 to 10 carbon atoms, represents in each case optionally halogen-, C₁-C₆-alkyl- or C₁-C₆-haloalkyl-substituted cycloalkyl or cycloalkylalkyl having in each case 3 to 6 carbon atoms in the cycloalkyl group and, if appropriate, 1 to 4 carbon atoms in the alkyl moiety, represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclylalkyl, where the heterocyclyl grouping in each case contains 1 to 6 carbon atoms and 1 to 4 nitrogen atoms and/or 1 oxygen atom and/or 1 sulfur atom and/or one (SO) grouping and/or one (SO₂) grouping and optionally additionally one (C═O) grouping and/or one (C═S) grouping and, if appropriate, the alkyl moiety contains 1 to 4 carbon atoms,
R⁴ and R⁵ are identical or different and independently of one another represent in each case optionally cyano-, halogen- or C₁-C₆-alkoxy-substituted alkyl, alkoxy, alkylthio, alkylamino or dialkylamino having in each case 1 to 6 carbon atoms in the alkyl groups, or represent in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl, phenoxy or phenylthio,
R⁶ represents hydrogen, represents optionally cyano-, halogen- or C₁-C₆-alkoxy-substituted alkyl having 1 to 6 carbon atoms or represents optionally cyano- or halogen-substituted alkenyl having 2 to 6 carbon atoms, and
R⁷ represents hydrogen, represents in each case optionally cyano-, halogen- or C₁-C₆-alkoxy-substituted alkyl or alkoxy having in each case 1 to 6 carbon atoms, represents optionally cyano- or halogen-substituted alkenyl having 2 to 6 carbon atoms, represents in each case optionally cyano-, halogen- or C₁-C₆-alkyl-substituted cycloalkyl or cycloalkylalkyl having in each case 3 to 6 carbon atoms in the cycloalkyl group and, if appropriate, 1 to 4 carbon atoms in the alkyl moiety, or represents in each case optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted aryl or arylalkyl having in each case 6 or 10 carbon atoms in the aryl group and, if appropriate, 1 to 4 carbon atoms in the alkyl moiety, or together with R⁶ represents optionally hydroxyl-, cyano- or C₁-C₆-alkyl-substituted C₂-C₆-alkanediyl in which optionally one methylene grouping is replaced by O (oxygen) or S (sulfur).

Preferred substituents or preferred ranges of the radicals present in the formula (IA) given above are defined below.
A′ preferably represents optionally cyano-, halogen-, C₁-C₅-alkoxy-, C₁-C₅-alkylthio-, C₁-C₅-alkylsulfinyl-, C₁-C₅-alkylsulfonyl- or C₁-C₅-alkoxy-carbonyl-substituted C₁-C₆-alkyl, represents in each case optionally cyano- or halogen-substituted C₂-C₆-alkenyl or C₂-C₆-alkynyl, represents in each case optionally cyano-, halogen-, C₁-C₄-alkyl-, C₁-C₄-haloalkyl-, C₁-C₄-alkoxy- or C₁-C₄-haloalkoxy-substituted C₃-C₅-cycloalkyl, C₇-C₈-cycloalkyl, C₅-C₆-cycloalkenyl or C₃-C₇-cycloalkyl-C₁-C₃-alkyl, represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl- or C₁-C₃-alkylenedioxy-substituted phenyl or phenyl-C₁-C₃-alkyl, represents in each case optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-halo-alkylthio-, C₁-C₅-alkoxy-carbonyl- or C₁-C₃-alkylenedioxy-substituted heterocyclyl or heterocyclyl-C₁-C₄-alkyl, where the heterocyclyl grouping in each case contains 1 to 5 carbon atoms and 1 to 3 nitrogen atoms and/or 1 oxygen atom and/or 1 sulfur atom and/or one (SO) grouping and/or one (SO₂) grouping and optionally additionally one (C═O) grouping and/or one (C═S) grouping, or represents the cyclohexyl grouping below

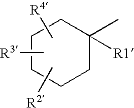

in which
R^{1′} represents hydrogen, cyano, C₁-C₅-alkyl or C₁-C₅-alkoxy-carbonyl,
R^{2′}, R^{3′} and R^{4′} are identical or different and individually represent hydrogen, halogen, C₁-C₅-alkyl, C₁-C₅-haloalkyl, C₁-C₅-alkoxy, C₁-C₅-haloalkoxy or benzyloxy, or two of these radicals (if adjacent) together represent C₃-C₅-alkanediyl which is optionally interrupted by 1 or 2 heteroatoms from the group consisting of N, O, S,
A furthermore preferably represents cyano-, halogen-, C₁-C₄-alkyl-, C₁-C₄-haloalkyl-, C₁-C₄-alkoxy- or C₁-C₄-haloalkoxy-substituted adamantyl or represents the cyclohexyl grouping below

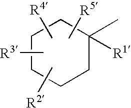

in which
R^{1′} represents hydrogen, cyano, C₁-C₅-alkyl or C₁-C₅-alkoxy-carbonyl,
R^{2′}, R^{3′}, R^{4′} and R^{5′} are identical or different and individually represent hydrogen, halogen, C₁-C₅-alkyl, C₁-C₅-haloalkyl, C₁-C₅-alkoxy, C₁-C₅-haloalkoxy, C₁-C₅-alkoxy-carbonyl or benzyloxy, or two of these radicals (if adjacent) together represent C₃-C₅-alkanediyl which is optionally interrupted by 1 or 2 heteroatoms from the group consisting of N, O, S,
Ar′ preferably represents mono- to pentasubstituted phenyl, except for 3,4-dimethoxyphenyl and 3,4-methylenedioxyphenyl, which are excluded by disclaimer, suitable substituents being—with the above disclaimer being taken into account—the following substituents:
   nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, phenyl (which contains one or more of the substituents listed above), phenoxy (which optionally contains one or more of the substituents listed above), phenylthio (which optionally contains one or more of the substituents listed above), phenyl-C₁-C₃-alkyl (which optionally contains one or more of the substituents listed above), phenyl-C₁-C₃-alkoxy (which optionally contains one or more of the substituents listed above), phenyl-C₁-C₃-alkylthio (which optionally contains one or more of the substituents listed above), heterocyclyl (which optionally contains one or more of the substituents listed above), heterocyclyloxy (which optionally contains one or more of the substituents listed above), heterocyclylthio (which optionally contains one or more of the substituents listed above), heterocyclyl-C₁-C₃-alkyl (which optionally contains one or more of the substituents listed above), heterocyclyl-C₁-C₃-alkoxy (which optionally contains one or more of the substituents listed above), heterocyclyl-C₁-C₃-alkylthio (which optionally contains one or more of the substituents listed above), where the heterocyclyl grouping in each case contains 1 to 5 carbon atoms and 1 to 3 nitrogen atoms and/or 1 oxygen atom and/or 1 sulfur atom and/or one (SO) grouping and/or one (SO₂) grouping and optionally additionally one (C═O) grouping and/or one (C═S) grouping,
R′ preferably represents hydrogen or one of the groupings below

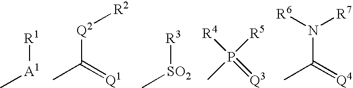

where
A¹, Q¹, Q², Q³, Q⁴, R¹, R², R³, R⁴, R⁵, R⁶ and R⁷ have the particularly preferred meanings given above in the definition of the compounds of the formula (I).
A′ particularly preferably represents in each case optionally cyano-, fluorine-, chlorine-, bromine-, methoxy-, ethoxy-, n- or i-propoxy-, n-, i-, s- or t-butoxy, methylthio-, ethylthio-, n- or i-propylthio-, n-, i-, s- or t-butylthio-, methylsulfinyl-, ethylsulfinyl-, n- or i-propylsulfinyl-, methylsulfonyl-, ethylsulfonyl-, methoxycarbonyl-, ethoxycarbonyl-, n- or i-propoxycarbonyl-substituted methyl, ethyl, n- or i-propyl, n-, i-, s- or t-butyl, represents in each case optionally cyano-, fluorine, chlorine- or bromine-substituted ethenyl, propenyl, butenyl, ethynyl, propynyl or butynyl, represents in each case optionally cyano-, fluorine-, chlorine-, bromine-, methyl- or ethyl-substituted cyclopropyl, cyclobutyl, cyclopentyl, cycloheptyl, cyclooctyl, cyclobutenyl, cyclopentenyl, cyclohexenyl, cyclopropylmethyl, cyclobutylmethyl, cyclopentylmethyl or cyclohexylmethyl, represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, difluoroethoxy-, trifluoroethoxy-, methylthio-, ethylthio-, n- or i-propylthio-, n-, i-, s- or t-butylthio-, difluoromethylthio-, trifluoromethylthio-, chlorodifluoromethylthio-, methoxy-carbonyl-, ethoxycarbonyl-, n- or i-propoxycarbonyl-, n-, i-, s- or t-butoxycarbonyl-, methylenedioxy- or dimethylenedioxy-substituted phenyl, benzyl or phenylethyl, represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n, i-, s- or t-butoxy-, fluoromethoxy-, difluoromethoxy-, trifluoromethoxy-, chlorodifluoromethoxy-, fluoroethoxy-, difluoroethoxy-, trifluoroethoxy-, methylthio-, ethylthio-, n- or i-propylthio-, n-, i-, s- or t-butylthio-, difluoromethylthio-, trifluoromethylthio-, chlorodifluoromethylthio-, methoxycarbonyl-, ethoxycarbonyl-, n- or i-propoxycarbonyl-, n-, i-, s- or t-butoxycarbonyl-, methylenedioxy- or dimethylenedioxy-substituted heterocyclyl, heterocyclylmethyl or heterocyclylethyl, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl, or represents the cyclohexyl grouping below

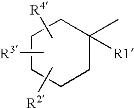

in which
R^{1′} represents hydrogen, cyano, methyl, ethyl, n- or i-propyl, n-, i-, s- or t-butyl, methoxy-carbonyl, ethoxycarbonyl, n- or i-propoxycarbonyl, n-, i-, s- or t-butoxycarbonyl,
R^{2′}, R^{3′} and R^{4′} are identical or different and individually represent hydrogen, fluorine, chlorine, bromine, methyl, ethyl, n- or i-propyl, n-, i-, s- or t-butyl, trifluoromethyl, methoxy, ethoxy, n- or i-propoxy, n-, i-, s- or t-butoxy or benzyloxy, or two of these radicals (if adjacent) together represent propane-1,3-diyl (trimethylene) or butane-1,4-diyl (tetramethylene) which is optionally interrupted by one heteroatom from the group consisting of N, O, S.
A′ furthermore particularly preferably represents in each case optionally cyano-, fluorine-, chlorine-, bromine-, methoxy-, ethoxy-, n- or i-propoxy-, n-, i-, s- or t-butoxy-, methylthio-, ethylthio-, n- or i-propylthio-, n-, i-, s- or t-butylthio-, methylsulfinyl-, ethylsulfinyl-, n- or i-propylsulfinyl-, methylsulfonyl-, ethylsulfonyl-, methoxycarbonyl-, ethoxycarbonyl-, n- or i-propoxycarbonyl-substituted n-, i-, s-, t- or neo-pentyl, represents optionally cyano-, fluorine-, chlorine-, bromine-, methyl- or ethyl-substituted adamantyl, represents optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, fluoro-methoxy-, difluoromethoxy-, trifluoromethoxy-, chlorodifluoromethoxy-, fluoroethoxy-, difluoroethoxy-, trifluoroethoxy-, methylthio-, ethylthio-, n- or i-propylthio-, n-, i-, s- or t-butylthio-, difluoromethylthio-, trifluoromethylthio-, chlorodifluoromethylthio-, methoxy-carbonyl-, ethoxycarbonyl-, n- or i-propoxycarbonyl-, n-, i-, s- or t-butoxycarbonyl-, methylenedioxy- or dimethylenedioxy-substituted pyranyl, piperidinyl, 1-methylbenzyl or 1,1-dimethylbenzyl, or represents the cyclohexyl grouping below

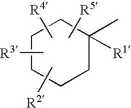

in which
R^{1′} represents hydrogen, cyano, methyl, ethyl, n- or i-propyl, n-, i-, s- or t-butyl, methoxy-carbonyl, ethoxycarbonyl, n- or i-propoxycarbonyl, n-, i-, s- or t-butoxycarbonyl,
R^{2′}, R^{3′}, R^{4′} and R^{5′} are identical or different and individually represent hydrogen, fluorine, chlorine, bromine, methyl, ethyl, n- or i-propyl, n-, i-, s- or t-butyl, trifluoromethyl, methoxy, ethoxy, n- or i-propoxy, n-, i-, s- or t-butoxy, methoxycarbonyl, ethoxycarbonyl, n- or i-propoxycarbonyl or benzyloxy, or two of these radicals (if adjacent) together represent propane-1,3-diyl (trimethylene) or butane-1,4-diyl (tetramethylene) which is optionally interrupted by one heteroatom from the group consisting of N, O, S.
Ar′ particularly preferably represents mono- to tetrasubstituted phenyl, except for 3,4-dimethoxyphenyl and 3,4-methylenedioxyphenyl, which are excluded by disclaimer, suitable substituents being—with the above disclaimer being taken into account—the following substituents:
   nitro, cyano, carbamoyl, thiocarbamoyl, fluorine, chlorine, bromine, methyl, ethyl, n- or i-propyl, n-, i-, s- or t-butyl, trifluoromethyl, ethoxy, n- or i-propoxy, n-, i-, s- or t-butoxy, difluoromethoxy, trifluoromethoxy, methoxymethyl, ethoxymethyl, methoxyethyl, ethoxyethyl, methylthio, ethylthio, n- or i-propylthio, n-, i-, s- or t-butylthio, difluoromethyl-thio, trifluoromethylthio, chlorodifluoromethylthio, methylsulfinyl, ethylsulfinyl, n- or i-propylsulfinyl, trifluoromethylsulfinyl, methylsulfonyl, ethylsulfonyl, trifluoromethyl-sulfonyl, phenyl (which contains one or more of the substituents listed above), phenoxy (which optionally contains one or more of the substituents listed above), phenylthio (which optionally contains one or more of the substituents listed above), benzyl or phenylethyl (which in each case optionally contains one or more of the substituents listed above), phenyl-methoxy (which optionally contains one or more of the substituents listed above), phenyl-methylthio (which optionally contains one or more of the substituents listed above), hetero-cyclyl (which optionally contains one or more of the substituents listed above), heterocyclyl-oxy (which optionally contains one or more of the substituents listed above), heterocyclylthio (which optionally contains one or more of the substituents listed above), heterocyclylmethyl (which optionally contains one or more of the substituents listed above), heterocyclylmethoxy (which optionally contains one or more of the substituents listed above), hetero-cyclylmethylthio (which optionally contains one or more of the substituents listed above), where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl.
R′ particularly preferably represents hydrogen or one of the groupings below

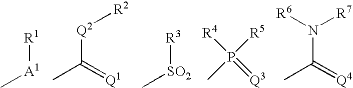

where
A¹, Q¹, Q², Q³, Q⁴, R¹, R², R³, R⁴, R⁵, R⁶ and R⁷ have the very particularly preferred meaning given above in the definition of the compounds of the formula (I).
A′ very particularly preferably represents in each case optionally cyano-, fluorine-, chlorine-, methoxy-, ethoxy-, n- or i-propoxy-, methylthio-, ethylthio-, n- or i-propylthio-, methyl-sulfinyl-, ethylsulfinyl-, methylsulfonyl-, ethylsulfonyl-, methoxycarbonyl- or ethoxy-carbonyl-substituted ethyl, n- or i-propyl, n-, i-, s- or t-butyl, represents in each case optionally cyano-, fluorine-, chlorine- or bromine-substituted propenyl, butenyl, propynyl or butynyl, represents in each case optionally cyano-, fluorine-, chlorine-, methyl-, ethyl-, methoxy- or ethoxy-substituted cyclopropyl, cyclobutyl, cyclopentyl, cycloheptyl, cyclopentenyl or cyclohexenyl, represents phenyl, benzyl, phenylethyl or 1,1-dimethylbenzyl, each of which is optionally mono- to trisubstituted by nitro, cyano, fluorine, chlorine, bromine, methyl, ethyl, n- or i-propyl, n-, i-, s- or t-butyl, trifluoromethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, methylthio, ethylthio, n- or i-propylthio, difluoromethylthio, trifluoromethylthio, chlorodifluoromethylthio, methoxycarbonyl, ethoxycarbonyl, n- or i-propoxycarbonyl, methylenedioxy or dimethylenedioxy, represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, fluoromethoxy-, difluoromethoxy-, trifluoromethoxy-, chlorodifluoromethoxy-, fluoro-ethoxy-, difluoroethoxy-, trifluoroethoxy-, methylthio-, ethylthio-, n- or i-propylthio-, difluoromethylthio-, trifluoromethylthio-, chlorodifluoromethylthio-, methoxycarbonyl-, ethoxycarbonyl-, n- or i-propoxycarbonyl-, methylenedioxy- or dimethylenedioxy-substituted heterocyclyl, heterocyclylmethyl or heterocyclylethyl, where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, piperidinyl, pyridinyl, pyrimidinyl, pyrazinyl, pyridazinyl, or represents the cyclohexyl grouping below

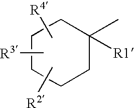

in which
R^{1′} represents hydrogen, cyano, methyl, ethyl, n- or i-propyl, methoxycarbonyl, ethoxy-carbonyl, n- or i-propoxycarbonyl,
R^{2′}, R^{3′} and R^{4′} are identical or different and individually represent hydrogen, fluorine, chlorine, bromine, methyl, ethyl, n- or i-propyl, methoxy, ethoxyl, n- or i-propoxy or benzyloxy, or two of these radicals (if adjacent) together represent propane-1,3-diyl (trimethylene) or butane-1,4-diyl (tetramethylene) which is optionally interrupted by one heteroatom from the group consisting of N, O, S.
A′ furthermore very particularly preferably represents optionally cyano-, fluorine-, chlorine-, methoxy-, ethoxy-, n- or i-propoxy-, methylthio-, ethylthio-, n- or i-propylthio-, methyl-sulfinyl-, ethylsulfinyl-, methylsulfonyl-, ethylsulfonyl-, methoxycarbonyl-, ethoxycarbonyl- or t-butoxycarbonyl-substituted n-, i-, s-, t- or neo-pentyl, cyclopropylmethyl, piperidinyl or pyranyl, represents optionally cyano, fluorine-, chlorine-, methyl-, ethyl-, methoxy- or ethoxy-substituted adamantyl or represents the cyclohexyl grouping below

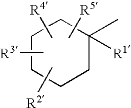

in which
R^{1′} represents hydrogen, cyano, methyl, ethyl, n- or i-propyl, methoxycarbonyl, ethoxy-carbonyl, n- or i-propoxycarbonyl,
R^{2′}, R^{3′}, R^{4′} and R^{5′} are identical or different and individually represent hydrogen, fluorine, chlorine, bromine, methyl, ethyl, n- or i-propyl, trifluoromethyl, trifluoromethoxy, methoxycarbonyl, ethoxycarbonyl, n- or i-propoxycarbonyl, methoxy, ethoxy, n- or i-propoxy, n-, i-, s- or t-butoxy or benzyloxy, or two of these radicals (if adjacent) together represent propane-1,3-diyl (trimethylene) or butane-1,4-diyl (tetramethylene) which is optionally interrupted by one heteroatom from the group consisting of N, O, S.
Ar′ very particularly preferably represents mono- to trisubstituted phenyl, possible substituents being the following:
   nitro, cyano, carbamoyl, thiocarbamoyl, fluorine, chlorine, bromine, methyl, ethyl, n- or i-propyl, n-, i-, s- or t-butyl, trifluoromethyl, ethoxy, n- or i-propoxy, n-, i-, s- or t-butoxy, difluoromethoxy, trifluoromethoxy, methoxymethyl, ethoxymethyl, methoxyethyl, ethoxyethyl, methylthio, ethylthio, n- or i-propylthio, n-, i-, s- or t-butylthio, difluoromethyl-thio, trifluoromethylthio, chlorodifluoromethylthio, methylsulfinyl, ethylsulfinyl, n- or i-propylsulfinyl, trifluoromethylsulfinyl, methylsulfonyl, ethylsulfonyl, trifluoromethyl-sulfonyl, phenyl (which contains one or more of the substituents listed above), phenoxy (which optionally contains one or more of the substituents listed above), phenylthio (which optionally contains one or more of the substituents listed above), benzyl or phenylethyl (which in each case optionally contains one or more of the substituents listed above), phenyl-methoxy (which optionally contains one or more of the substituents listed above), phenyl-methylthio (which optionally contains one or more of the substituents listed above), hetero-cyclyl (which optionally contains one or more of the substituents listed above), heterocyclyl-oxy (which optionally contains one or more of the substituents listed above), heterocyclylthio (which optionally contains one or more of the substituents listed above), heterocyclylmethyl (which optionally contains one or more of the substituents listed above), heterocyclylmethoxy (which optionally contains one or more of the substituents listed above), hetero-cyclylmethylthio (which optionally contains one or more of the substituents listed above), where the heterocyclyl grouping is in each case selected from the group consisting of furyl, tetrahydrofuryl, thienyl, pyrrolyl, pyrrolinyl, pyrrolidinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl.
R′ very particularly preferably represents hydrogen or one of the groupings below

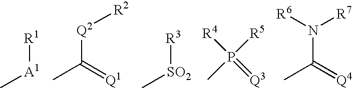

where
A¹, Q¹, Q², Q³, Q⁴, R¹, R², R³, R⁴, R⁵, R⁶ and R⁷ have the very particularly preferred meaning given above in the definition of the compounds of the formula (I).
A′ especially preferably represents in each case optionally fluorine- or ethoxycarbonyl-substituted ethyl, n-propyl, i-propyl, t-butyl or propenyl, represents phenyl, benzyl or 1,1-dimethylbenzyl, each of which is optionally mono- to trisubstituted by fluorine, chlorine, methyl, trifluoromethyl, ethoxy or trifluoromethoxy, represents heterocyclyl which is in each case optionally substituted by trifluoromethyl or ethoxy, where the heterocyclyl grouping is in each case selected from the group consisting of thiazolyl or pyridazinyl, or represents the heterocyclyl grouping below

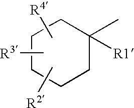

in which
R^{1′} represents hydrogen, cyano, methoxycarbonyl or ethoxycarbonyl,
R^{2′}, R^{3′} and R^{4′} are identical or different and individually represent hydrogen, methyl, i-propyl or methoxy.
A′ furthermore especially preferably represents pyranyl, cyclopropylmethyl, neo-pentyl, represents adamantyl, represents t-butoxycarbonyl-substituted piperidinyl or represents the heterocyclyl grouping below

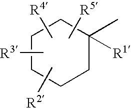

in which
R^{1′} represents hydrogen, methoxycarbonyl or ethoxycarbonyl,
R^{2′}, R^{3′}, R^{4′} and R^{5′} are identical or different and individually represent hydrogen, trifluoromethyl, methoxycarbonyl, ethoxycarbonyl or n-butoxy,
Ar′ especially preferably represents mono- to trisubstituted phenyl, the following substituents being possible:
   nitro, cyano, fluorine, chlorine, bromine, methyl, trifluoromethyl, trifluoromethoxy or phenyl (which contains one or more of the substituents listed above),
Ar′ furthermore especially preferably represents methoxy- or iodine-substituted phenyl.
R especially preferably represents hydrogen or one of the groupings below

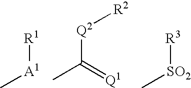

where
A¹ represents a carbonyl group (C═O) or represents methylene,
Q¹, Q² each represent O,
R¹ represents methyl, t-butyl or represents optionally chlorine-substituted phenyl,
R¹ furthermore represents 1,1,2-trimethylpropyl or ethoxy,
R² represents i-propyl,
R³ represents optionally methyl-substituted phenyl.

Saturated or unsaturated hydrocarbon radicals, such as alkyl or alkenyl, may in each case be straight-chain or branched as far as this is possible, including in combination with heteroatoms, such as, for example, in alkoxy.

Optionally substituted radicals may be mono- or polysubstituted, where in the case of polysubstitution the substituents can be identical or different.

Halogen-substituted radicals, such as, for example, haloalkyl, are mono- or polyhalogenated. In the case of multiple halogenation, the halogen atoms may be identical or different. Here, halogen represents fluorine, chlorine, bromine and iodine, in particular fluorine, chlorine and bromine.

Preference according to the invention is given to novel compounds of the formula (IA) which contain a combination of the meanings given above as being preferred for the radicals A′, Ar′ and R′.

Particular preference according to the invention is given to novel compounds of the formula (IA) which contain a combination of the meanings given above as being particularly preferred for the radicals A′, Ar′ and R′.

Very particular preference according to the invention is given to novel compounds of the formula (IA) which contain a combination of the meanings given above as being very particularly preferred for the radicals A′, Ar′ and R′.

Special preference according to the invention is given to novel compounds of the formula (IA) which contain a combination of the meanings given above as being especially preferred for the radicals A′, Ar′ and R′.

However, the general or preferred radical definitions and explanations given above can also be combined with one another as desired, i.e. between the respective ranges and preferred ranges. They apply both to the end products and, correspondingly, to precursors and intermediates.

Depending on the nature of the substituents, the novel compounds of the formula (IA) may be present as geometrical and/or optical isomers or corresponding isomer mixtures of varying composition. What is claimed according to the invention are both the pure isomers and the isomer mixtures.

The novel substituted 1H-pyrrole-2,5-diones of the formula (IA)—and analogously also the 1H-pyrrole-2,5-diones of the formula (I), some of which are known—are obtained when arylmethyl-carboxamides of the formula (II)

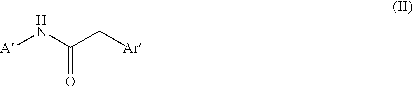

in which
A′ and Ar′ are as defined above PS are reacted with oxalic acid diesters of the formula (III)

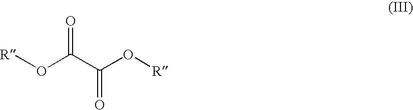

in which
R″ represents alkyl,
if appropriate in the presence of a reaction auxiliary and if appropriate in the presence of a diluent, and the resulting 3-hydroxy-1H-pyrrole-2,5-diones of the formula (IB)

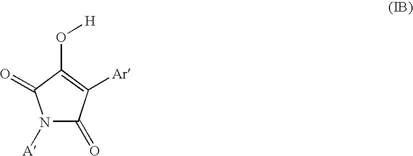

are, if appropriate, reacted with compounds of the formula (IV)

X—R′ (IV)

in which
R′ is as defined above and
X represents halogen,
if appropriate in the presence of a reaction auxiliary and if appropriate in the presence of a diluent.

Using, for example, N-methyl-2,4-dichlorophenylacetamide and dimethyl oxalate as starting materials, the course of the reaction in the process according to the invention can be illustrated by the formula scheme below:

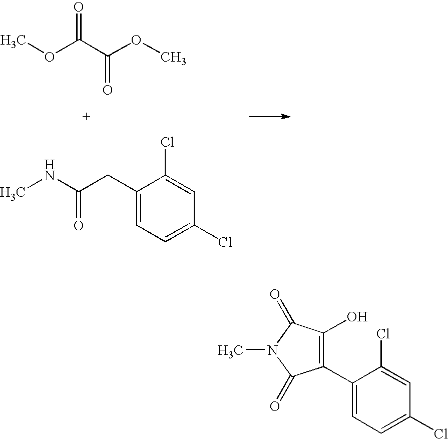

The formula (II) provides a general definition of the arylmethylcarboxamides to be used as starting material in the process according to the invention for preparing the compounds of the general formula (I) or (IB). In the general formula (II), A′ and Ar′ preferably, particularly preferably and very particularly preferably have those meanings which have already been mentioned above, in connection with the description of the compounds of the general formula (I) according to the invention, as being preferred, particularly preferred, very particularly preferred and especially preferred, respectively, for A′ and Ar′.

The starting materials of the general formula (II) are known and/or can be prepared by processes known per se (cf. EP-A-456063, EP-A-595130, EP-A-596298, EP-A-613885, U.S. Pat. No. 4,455,164, U.S. Pat. No. 5,622,917, WO-95/01358, WO-95/20572, WO-95/26954, WO-96/25395, WO-96/35664, WO97/02243, WO-97/28133, WO-98/06721, WO98/25928). The arylmethylcarboxamides of the formula (I) are generally obtained by reacting corresponding carbonyl chlorides with amines, if appropriate in the presence of acid binders, such as, for example, triethylamine, or by reacting corresponding carboxylic acids with amines in the presence of dehydrating agents (cf., for example, J. March, Advanced Organic Chemistry, 3rd Edition 1985, p. 370 ff.).

The formula (III) provides a general definition of the oxalic acid diesters further to be used as starting materials in the process according to the invention for preparing the compounds of the general formula (I). In the general formula (III), R″ preferably represents alkyl having 1 to 4 carbon atoms, in particular methyl or ethyl.

The starting materials of the general formula (III) are known organic chemicals for synthesis.

The formula (IV) provides a general definition of the compounds further to be used as starting materials in the process according to the invention for preparing the compounds of the general formula (I) or (IA). In the general formula (IV), R′ preferably, particularly preferably, very particularly preferably and especially preferably has those meanings which have already been mentioned above, in connection with the description of the compounds of the general formula (I) according to the invention, as being preferred, particularly preferred, very particularly preferred and especially preferred for R′.

The starting materials of the general formula (IV) are known organic chemicals for synthesis.

The process according to the invention for preparing the compounds of the general formula (I) or (IA) or (IB) is preferably carried out using one or more reaction auxiliaries. Suitable reaction auxiliaries for the process according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, cesium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2,2,2]octane (DABCO), 1,5-diazabicyclo[4,3,0]non-5-ene (DBN), or 1,8-diazabicyclo[5,4,0]undec-7-ene (DBU).

The process according to the invention for preparing the compounds of the general formula (I) or (IA) or (IB) is preferably carried out using one or more diluents. Suitable diluents for carrying out the process according to the invention are especially inert organic solvents. These include in particular aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulfoxides, such as dimethyl sulfoxide, alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the process according to the invention, the reaction temperatures may be varied within a relatively wide range. In general, the process is carried out at temperatures between −30° C. and +150° C., preferably between 0° C. and 120° C.

The process of the invention is generally carried out under atmospheric pressure. It is, however, also possible to carry out the process of the invention under elevated or reduced pressure—in general between 0.1 bar and 10 bar.

For the implementation of the process of the invention the starting materials are generally used in approximately equimolar amounts. It is, however, also possible to use one of the components in a relatively large excess. The reaction is generally carried out in a suitable diluent in the presence of a reaction auxiliary, and the reaction mixture is generally stirred at the required temperature for a number of hours. Working up is carried out in accordance with typical methods (cf. the Preparation Examples).

The active compounds of the invention, in combination with good plant tolerance and favorable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Strongyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Aphelenchoides spp., Bursaphelenchus spp., Ditylenchus dipsaci, Globodera spp., Heterodera spp., Longidorus spp., Meloidogyne spp., Pratylenchus spp., Radopholus similis, Trichodorus spp., Tylenchulus semipenetrans, Xiphinema spp.

The compounds of the formula (I) according to the invention have in particular excellent activity against aphids (for example Aphis fabae, Myzus persicae), beetle larvae (for example Phaedon cochleariae), butterfly caterpillars (for example Plutella xylostella, Spodoptera frugiperda) and nematodes (for example Meloidogyne incognita).

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, habitat or storage space by the customary treatment methods, for example by immersion, spraying, evaporation, fogging, scattering, painting on, injection and, in the case of propagation material, in particular in the case of seeds, also by applying one or more coats.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers.

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulfoxide, and also water.

Suitable solid carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as sawdust, coconut shells, corn cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolysates; suitable dispersants are for example ligninsulfite waste liquors and methylcellulose. Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances, herbicides, safeners, fertilizers or semiochemicals.

Particularly favorable mixing components are, for example, the following compounds:

### Fungicides:

2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetylsodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro[4.5]decan-3-amine; sodium tetrathiocarbonate; and also copper salts and preparations, such as Bordeaux mixture; copper hydroxide; copper naphthenate; copper oxychloride; copper sulfate; cufraneb; copper oxide; mancopper; oxine-copper.

### Bactericides:

bronopol, dichlorophen, nitrapyrin, nickel dimethyldithiocarbamate, kasugamycin, octhilinone, furan-carboxylic acid, oxytetracyclin, probenazole, streptomycin, tecloftalam, copper sulfate and other copper preparations.

### Insecticides/Acaricides/Nematicides:

### Acetylcholine Esterase (AChE) Inhibitors

### 1.1 Carbamates,

for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, prome-carb, propoxur, thiodicarb, thiofanox, trimethacarb, XMC, xylylcarb, triazamate

### 1.2 Organophosphates,

for example acephate, azamethiphos, azinphos (-methyl, -ethyl), bromophos-ethyl, bromfen-vinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfen-vinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

### Sodium Channel Modulators/Voltage-Dependent Sodium Channel Blockers

### 2.1 Pyrethroids,

for example acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaporthrin, cis-cypernethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, perrnethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)
DDT

### 2.2 Oxadiazines,

for example indoxacarb

### Acetylcholine Receptor Agonists/Antagonists

### 3.1 Chloronicotinyls,

for example acetamiprid, clothianidin, dinotefuran, imidacloprid, nitenpyram, nithiazine, thiacloprid, thiamethoxam

### 3.2 Nicotine, Bensultap, Cartap

### Acetylcholine Receptor Modulators

### 4.1 Spinosyns,

for example spinosad

### GABA-Controlled Chloride Channel Antagonists

### 5.1 Organochlorines,

for example camphechlor, chlordane, endosulfan, gamma-HCH, HCH, heptachlor, lindane,
methoxychlor

### 5.2 Fiproles,

for example acetoprole, ethiprole, fipronil, pyrafluprole, pyriprole, vaniliprole

### Chloride Channel Activators

### 6.1 Mectins,

for example avermectin, emamectin, emamectin-benzoate, ivermectin, milbemycin

### Juvenile Hormone Mimetics,

for example diofenolan, epofenonane, fenoxycarb, hydroprene, kinoprene, methoprene, pyriproxifen, triprene

### Ecdysone Agonists/Disruptors

### 8.1 Diacylhydrazines,

for example chromafenozide, halofenozide, methoxyfenozide, tebufenozide

### Chitin Biosynthesis Inhibitors

### 9.1 Benzoylureas,

for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron

### 9.2 Buprofezin

### 9.3 Cyromazine

### Oxidative Phosphorylation Inhibitors, ATP Disruptors

### 10.1 Diafenthiuron

10.2 Organotin compounds,
for example azocyclotin, cyhexatin, fenbutatin-oxide

### Oxidative Phosphorylation Decouplers Acting by Interrupting the H-Proton Gradient

### 11.1 Pyrroles,

for example chlorfenapyr

### 11.2 Dinitrophenols,

for example binapacyrl, dinobuton, dinocap, DNOC

### Page-I Electron Transport Inhibitors

### 12.1 METIs,

for example fenazaquin, fenpyroximate, pyrimidifen, pyridaben, tebufenpyrad, tolfenpyrad

### 12.2 Hydramethylnon

### 12.3 Dicofol

### Page-II Electron Transport Inhibitors

Rotenone

### Page-III Electron Transport Inhibitors

Acequinocyl, fluacrypyrim

### Microbial Disruptors of the Insect Gut Membrane

Bacillus thuringiensis strains

### Fat Synthesis Inhibitors

tetronic acids,
for example spirodiclofen, spiromesifen
tetramic acids,
for example spirotetramat (CAS Reg. No.: 203313-25-1) and 3-(2,5-dimethylphenyl)-8-methoxy-2-oxo-1-azaspiro[4.5]dec-3-en-4-yl ethyl carbonate (aka: carbonic acid, 3-(2,5-dimethylphenyl)-8-methoxy-2-oxo-1-azaspiro[4.5]dec-3-en-4-yl ethyl ester, CAS Reg. No.: 382608-10-8)
carboxamides,
for example flonicamid
octopaminergic agonists,
for example amitraz

### Inhibitors of Magnesium-Stimulated ATPase,

propargite
benzoic acid dicarboxamides,
for example flubendiamides
nereistoxin analogs
for example thiocyclam hydrogen oxalate, thiosultap-sodium

### Biologicals, Hormones or Pheromones

azadirachtin, Bacillus spec., Beauveria spec., codlemone, Metarrhizium spec., Paecilomyces spec., thuringiensin, Verticillium spec.
   Active Compounds with Unknown or Unspecific Mechanisms of Action

### 23.1 Fumigants,

for example aluminum phosphide, methyl bromide, sulfuryl fluoride

### 23.2 Antifeedants,

for example cryolite, flonicamid, pymetrozine
   23.3 Mite growth inhibitors,
for example clofentezine, etoxazole, hexythiazox
   23.4 Amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin

A mixture with other known active compounds, such as herbicides, fertilizers, growth regulators, safeners, semiochemicals, or else with agents for improving the plant properties, is also possible. When used as insecticides, the active compounds according to the invention can furthermore be pre-sent in their commercially available formulations and in the use forms, prepared from these formulations, as a mixture with synergistic agents. Synergistic agents are compounds which increase the action of the active compounds, without it being necessary for the synergistic agent added to be active itself.

When used as insecticides, the active compounds according to the invention can furthermore be pre-sent in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compounds are employed in a customary manner appropriate for the use forms.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetic Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The transgenic plants or plant cultivars (obtained by genetic engineering) which are preferably to be treated according to the invention include all plants which, by virtue of the genetic modification, received genetic material which imparted particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soybeans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soybeans, potatoes, cotton, tobacco and oilseed rape. Traits that are emphasized are in particular increased defense of the plants against insects, arachnids, nematodes and worms by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soybean varieties and potato varieties which are sold under the trade names YIELD GARD® (for example maize, cotton, soybeans), KnockOut® (for example maize), StarLink® (for example maize), Bollgard® (cotton), Nu-cotn® (cotton) and NewLeaf® (potato). Examples of herbicide-tolerant plants which may be mentioned are maize varieties, cotton varieties and soybean varieties which are sold under the trade names Roundup Ready® (tolerance to glyphosate, for example maize, cotton, soybean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants by nasal administration, by dermal use in the form, for example, of dipping or bathing, spraying, pouring on and spotting on, washing and powdering, and also with the aid of molded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, such as hulls, screens, nets, buildings, moorings and signaling systems, against fouling.

Furthermore, the compounds according to the invention, alone or in combination with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoides, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

### PREPARATION EXAMPLES

### Example 1

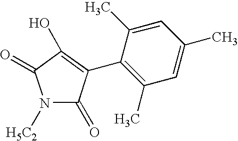

13.34 g (65 mmol) of N-ethyl-(2,4,6-trimethylphenyl)acetamide are initially charged in 200 ml of N,N-dimethylformamide, 10.2 g (70 mmol) of diethyl oxalate are added, 8.15 g (72.5 mmol) of potassium t-butoxide are added with ice-cooling, the mixture is stirred for 30 minutes, a further 8.15 g (72.5 mmol) of potassium t-butoxide are added and the mixture is stirred at room temperature (about 20° C.) for 6 hours.

For work-up, most of the solvent is distilled off under reduced pressure (on a rotary evaporator) and the crude product that remains is dissolved in water. Impurities are extracted with methyl t-butyl ether, the aqueous solution is then acidified using 6N hydrochloric acid and the resulting crystalline product is isolated by filtration with suction.

This gives 9.8 g (58% of theory) of 1-ethyl-3-hydroxy-4-(2,4,6-trimethylphenyl)-1H-pyrrole-2,5-dione of melting point 168° C.

### Example 2

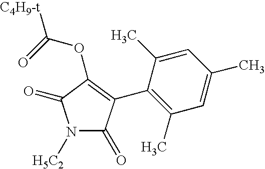

3.89 g (15 mmol) of 1-ethyl-3-hydroxy-4-(2,4,6-trimethylphenyl)-1H-pyrrole-2,5-dione (cf. Example 1) are dissolved in 50 ml of dichloromethane, 1.62 g (16 mmol) of triethylamine and a spatula tip of 4-dimethylaminopyridine are added and a solution of 1.93 g (16 mmol) of pivaloyl chloride in 10 ml of dichloromethane is added dropwise with ice-cooling. The reaction mixture is stirred at room temperature (about 20° C.) for 6 hours and then washed successively with aqueous sodium bicarbonate solution and 10% strength citric acid, dried with sodium sulfate and filtered. From the filtrate, the solvent is carefully distilled off under reduced pressure.

This gives 4.13 g (95% of theory) of 1-ethyl-3-pivaloyloxy-4-(2,4,6-trimethylphenyl)-1H-pyrrole-2,5-dione of melting point 91° C.

Examples of the compounds of the formula (I) to be prepared analogously—these are exclusively novel compounds also defined by formula (IA)—are listed in table 1 below.

TABLE 1
Examples of compounds of the formula (I)

| | |
|---|---|
| | (I) |
| 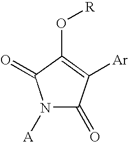 | |

| Ex. No. | A | Ar | R | Physical data |
|---|---|---|---|---|
| 3 | C₂H₅ | 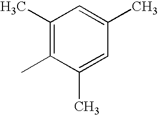 | CO—CH₃ | m.p.: 68° C. |
| 4 | CH₂—CH═CH₂ | 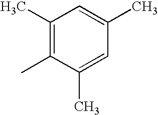 | H | m.p.: 138° C. |
| 5 | CH₂C₆H₅ | 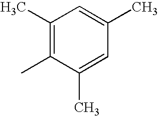 | H | m.p.: 185° C. |
| 6 | C₃H₇-i | 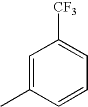 | H | m.p.: 183° C. |
| 7 | C(CH₃)₂C₆H₅ | 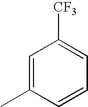 | H | m.p.: 250° C. |
| 8 | CH₂CF₃ | 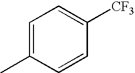 | H | |
| 9 | 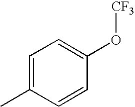 | 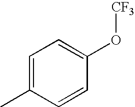 | H | logP = 4.12^{a)} |
| 10 | 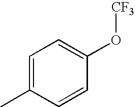 | 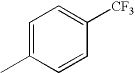 | H | logP = 3.64^{a)} |
| 11 | 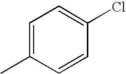 | 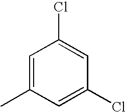 | H | logP = 3.27^{a)} |
| 12 | 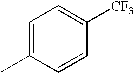 | 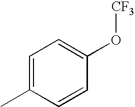 | H | logP = 3.97^{a)} |
| 13 | 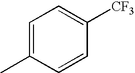 | 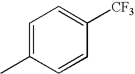 | H | logP = 3.51^{a)} |
| 14 | 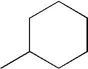 | 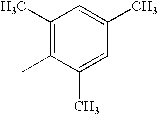 | H | m.p.: 189° C. |
| 15 | C₄H₉-t | 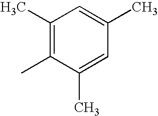 | H | m.p.: 152° C. |
| 16 | C(CH₃)₂C₆H₅ | 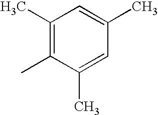 | H | m.p.: 90° C. |
| 17 | C₃H₇-i | 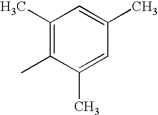 | H | m.p.: 171° C. |
| 18 | C₃H₇-i | 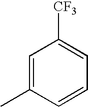 | CO—CH₃ | NMR (CDCl₃)d = 1.45 (d, 6H),2.42 (s, 3H), 4.42(m, 1H), 7.65 (m,2H), 8.05 (m,2H) ppm |
| 19 | C₃H₇-i | 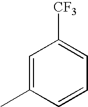 | CO—C₄H₉-t | NMR (CDCl₃)d = 1.40 (s, 9H),1.45 (d, 6H),4.40 (m, 1H),7.65 (m, 2H),8.10 (m, 2H)ppm |
| 20 | C₃H₇-i | 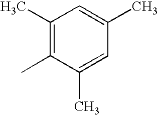 | CO—C(CH₃)₂CH(CH₃)₂ | logP = 5.87^{a)} |
| 21 | C₃H₇-i | 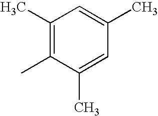 | CO—OC₃H₇-i | m.p.: 75° C. |
| 22 | 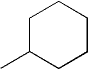 | 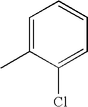 | H | logP = 3.17^{a)} |
| 23 | 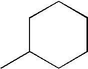 | 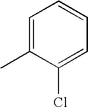 | CO—C₄H₉-t | logP = 5.39^{a)} |
| 24 | 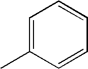 | 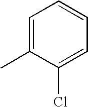 | H | logP = 2.43^{a)} |
| 25 | 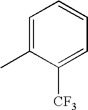 | 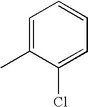 | H | logP = 2.60^{a)} |
| 26 | 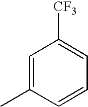 | 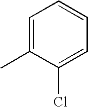 | H | logP = 3.04^{a)} |
| 27 | 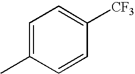 | 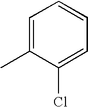 | H | logP = 3.10^{a)} |
| 28 | 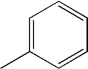 | 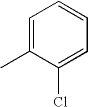 | CO—C₄H₉-t | logP = 4.37^{a)} |
| 29 | 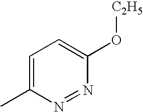 | 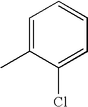 | H | logP = 1.82^{a)} |
| 30 | CH₂CF₃ | 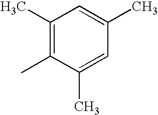 | H | |
| 31 | 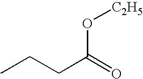 | 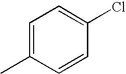 | H | |
| 32 | 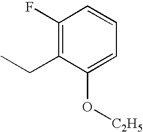 | 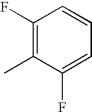 | H | |
| 33 | 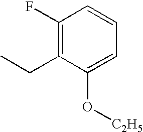 | 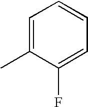 | H | |
| 34 | 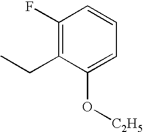 | 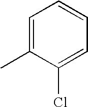 | H | |
| 35 | 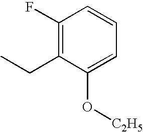 | 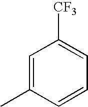 | H | |
| 36 | 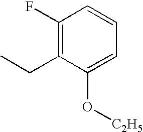 | 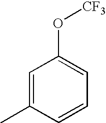 | H | |
| 37 | 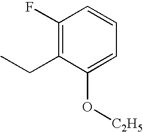 | 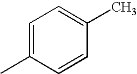 | H | |
| 38 | CH₂CF₃ | 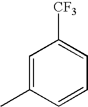 | H | logP = 2.70^{a)} |
| 39 | CH₂CF₃ | 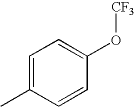 | H | logP = 3.04^{a)} |
| 40 | CH₂CF₃ | 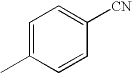 | H | logP = 1.94^{a)} |
| 41 | CH₂CF₃ | 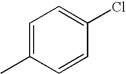 | H | logP = 2.82^{a)} |
| 42 | CH₂CF₃ | 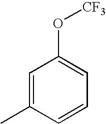 | H | logP = 2.82^{a)} |
| 43 | CH₂CF₃ | 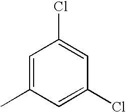 | H | logP = 2.67^{a)} |
| 44 | CH₂CH₂CF₃ | 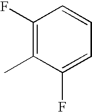 | H | logP = 2.14^{a)} |
| 45 | CH₂CH₂CF₃ | 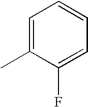 | H | |
| 46 | CH₂CH₂CF₃ | 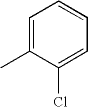 | H | |
| 47 | CH₂CH₂CF₃ | 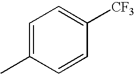 | H | logP = 3.06^{a)} |
| 48 | CH₂CH₂CF₃ | 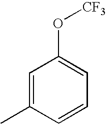 | H | logP = 3.30^{a)} |
| 49 | CH₂CH₂CF₃ | 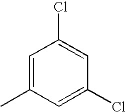 | H | logP = 2.94^{a)} |
| 50 | 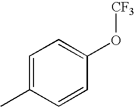 | 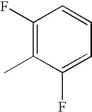 | H | logP = 2.80^{a)} |
| 51 | 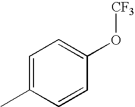 | 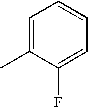 | H | logP = 3.13^{a)} |
| 52 | 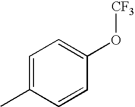 | 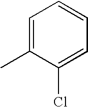 | H | logP = 3.27^{a)} |
| 53 | 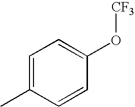 | 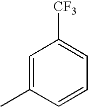 | H | logP = 3.75^{a)} |
| 54 | 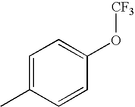 | 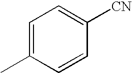 | H | logP = 2.70^{a)} |
| 55 | 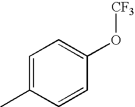 | 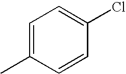 | H | logP = 3.83^{a)} |
| 56 | 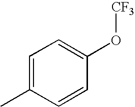 | 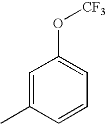 | H | |
| 57 | 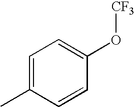 | 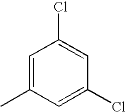 | H | logP = 3.56^{a)} |
| 58 | 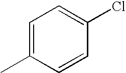 | 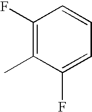 | H | logP = 2.45^{a)} |
| 59 | 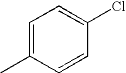 | 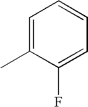 | H | logP = 2.76^{a)} |
| 60 | 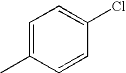 | 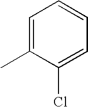 | H | logP = 2.89^{a)} |
| 61 | 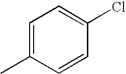 | 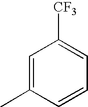 | H | logP = 3.45^{a)} |
| 62 | 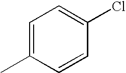 | 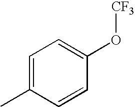 | H | logP = 3.80^{a)} |
| 63 | 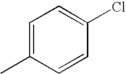 | 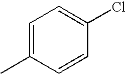 | H | logP = 3.51^{a)} |
| 64 | 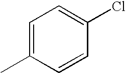 | 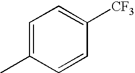 | H | logP = 3.35^{a)} |
| 65 | 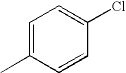 | 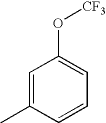 | H | logP = 3.59^{a)} |
| 66 | 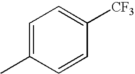 | 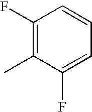 | H | logP = 3.48^{a)} |
| 67 | 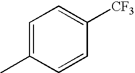 | 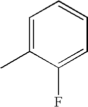 | H | logP = 3.01^{a)} |
| 68 | 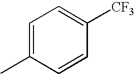 | 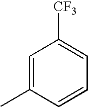 | H | logP = 3.59^{a)} |
| 69 | 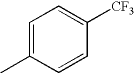 | 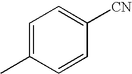 | H | logP = 2.80^{a)} |
| 70 | 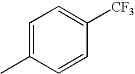 | 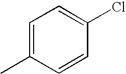 | H | logP = 3.70^{a)} |
| 71 | 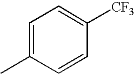 | 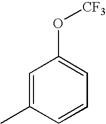 | H | logP = 3.75^{a)} |
| 72 | 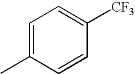 | 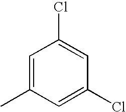 | H | logP = 3.42^{a)} |
| 73 | 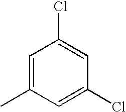 | 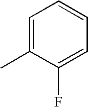 | H | logP = 3.20^{a)} |
| 74 | 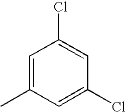 | 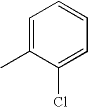 | H | |
| 75 | 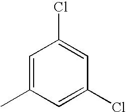 | 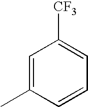 | H | logP = 3.78^{a)} |
| 76 | 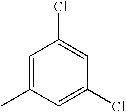 | 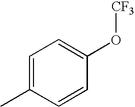 | H | logP = 4.15^{a)} |
| 77 | 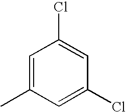 | 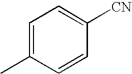 | H | logP = 2.80^{a)} |
| 78 | 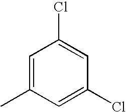 | 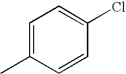 | H | logP = 3.94^{a)} |
| 79 | 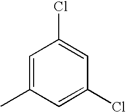 | 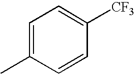 | H | logP = 3.75^{a)} |
| 80 | 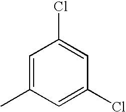 | 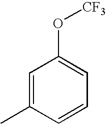 | H | logP = 3.94^{a)} |
| 81 | 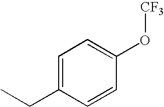 | 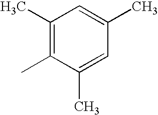 | H | logP = 4.24^{a)} |
| 82 | 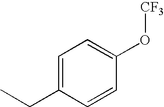 | 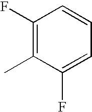 | H | logP = 3.16^{a)} |
| 83 | 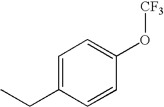 | 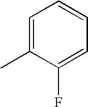 | H | logP = 3.45^{a)} |
| 84 | 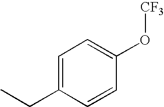 | 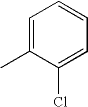 | H | logP = 3.56^{a)} |
| 85 | 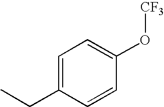 | 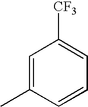 | H | logP = 4.15^{a)} |
| 86 | 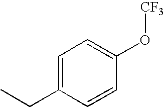 | 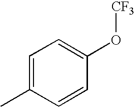 | H | logP = 4.48^{a)} |
| 87 | 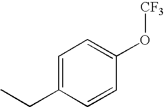 | 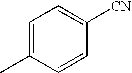 | H | logP = 2.85^{a)} |
| 88 | 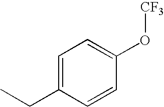 | 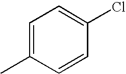 | H | logP = 4.27^{a)} |
| 89 | 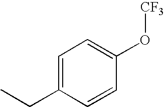 | 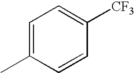 | H | logP = 4.03^{a)} |
| 90 | 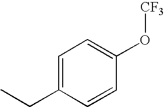 | 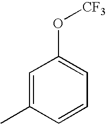 | H | logP = 4.30^{a)} |
| 91 | 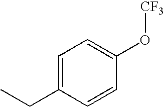 | 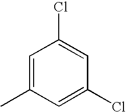 | H | logP = 4.12^{a)} |
| 92 | 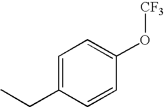 | 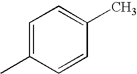 | H | logP = 4.27^{a)} |
| 93 | 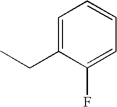 | 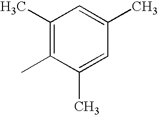 | H | logP = 3.51^{a)} |
| 94 | 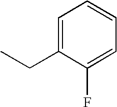 | 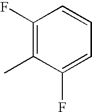 | H | logP = 2.49^{a)} |
| 95 | 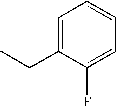 | 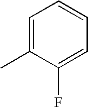 | H | logP = 2.82^{a)} |
| 96 | 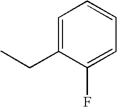 | 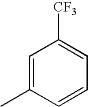 | H | logP = 3.48^{a)} |
| 97 | 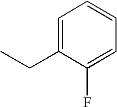 | 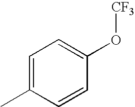 | H | logP = 3.83^{a)} |
| 98 | 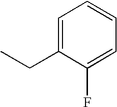 | 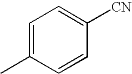 | H | logP = 2.31^{a)} |
| 99 | 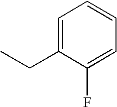 | 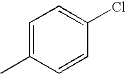 | H | logP = 3.56^{a)} |
| 100 | 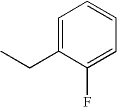 | 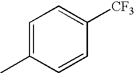 | H | logP = 3.40^{a)} |
| 101 | 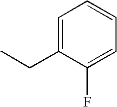 | 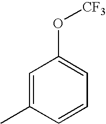 | H | logP = 3.64^{a)} |
| 102 | 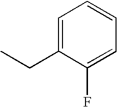 | 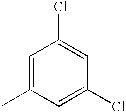 | H | logP = 3.48^{a)} |
| 103 | 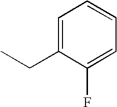 | 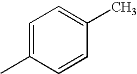 | H | logP = 3.51^{a)} |
| 104 | 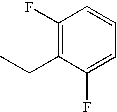 | 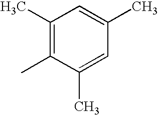 | H | logP = 3.48^{a)} |
| 105 | 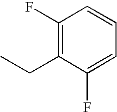 | 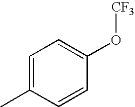 | H | |
| 106 | 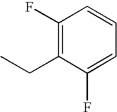 | 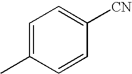 | H | |
| 107 | 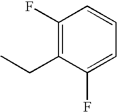 | 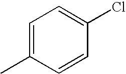 | H | |
| 108 | 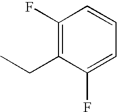 | 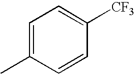 | H | |
| 109 | 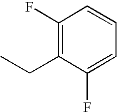 | 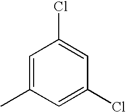 | H | logP = 3.20^{a)} |
| 110 | 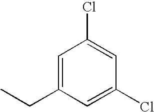 | 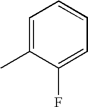 | H | logP = 3.56^{a)} |
| 111 | 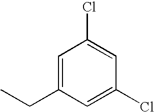 | 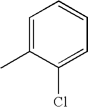 | H | logP = 3.67^{a)} |
| 112 | 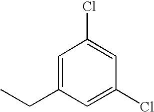 | 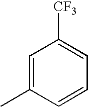 | H | logP = 4.27^{a)} |
| 113 | 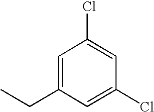 | 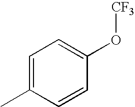 | H | logP = 4.67^{a)} |
| 114 | 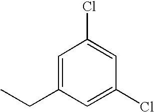 | 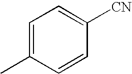 | H | logP = 2.94^{a)} |
| 115 | 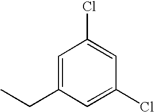 | 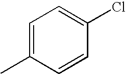 | H | logP = 4.48^{a)} |
| 116 | 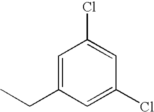 | 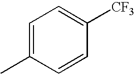 | H | logp = 4.18^{a)} |
| 117 | 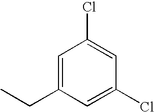 | 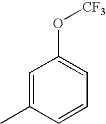 | H | logP = 4.45^{a)} |
| 118 | 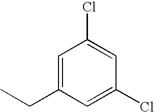 | 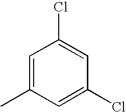 | H | logP = 4.21^{a)} |
| 119 | 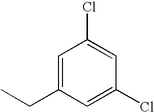 | 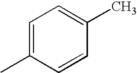 | H | logp = 4.42^{a)} |
| 120 | 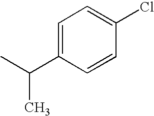 | 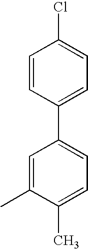 | H | logP = 4.94^{a)} |
| 121 | 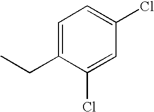 | 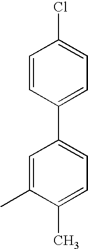 | H | logP = 5.03^{a)} |
| 122 | C₃H₇-i | 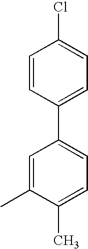 | H | logP = 4.08^{a)} |
| 123 | 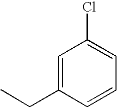 | 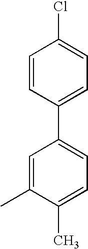 | H | logP = 4.61^{a)} |
| 124 | 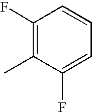 | 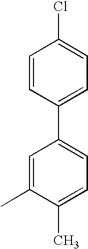 | H | logP = 3.85^{a)} |
| 125 | 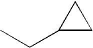 | 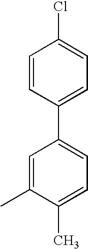 | H | logP = 4.08^{a)} |
| 126 | 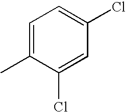 | 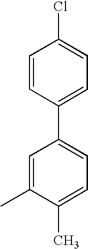 | H | logP = 4.51^{a)} |
| 127 | 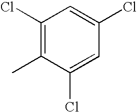 | 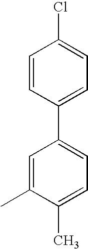 | H | logP = 4.75^{a)} |
| 128 | 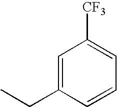 | 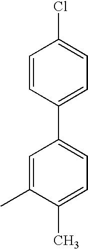 | H | logP = 4.65^{a)} |
| 129 | 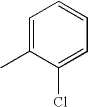 | 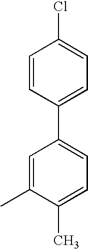 | H | logP = 4.03^{a)} |
| 130 | 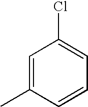 | 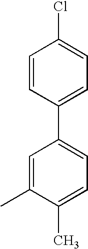 | H | |
| 131 | 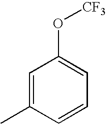 | 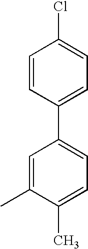 | H | |
| 132 | 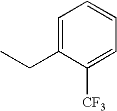 | 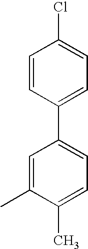 | H | |
| 133 | 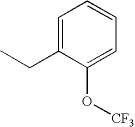 | 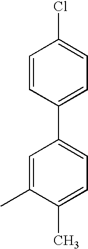 | H | logP = 4.75^{a)} |
| 134 | 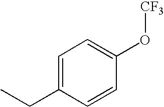 | 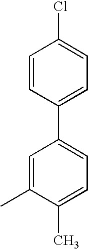 | H | logP = 4.84^{a)} |
| 135 | 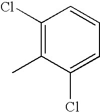 | 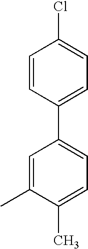 | H | logP = 4.18^{a)} |
| 136 | 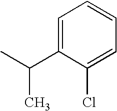 | 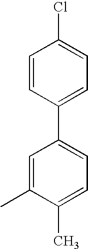 | H | logP = 4.75^{a)} |
| 137 | C₄H₉-t | 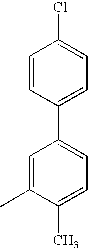 | H | logP = 4.51^{a)} |
| 138 | CH₂C(CH₃)₃ | 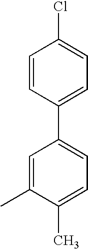 | H | logP = 4.61^{a)} |
| 139 | 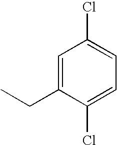 | 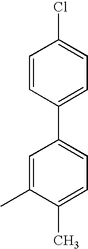 | H | logP = 4.84^{a)} |
| 140 | 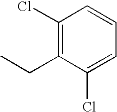 | 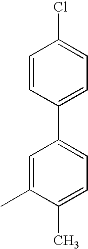 | H | logP = 4.70^{a)} |
| 141 | 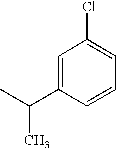 | 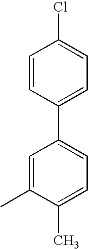 | H | logP = 4.84^{a)} |
| 142 | 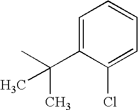 | 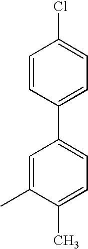 | H | logP = 4.80^{a)} |
| 143 | 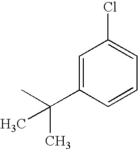 | 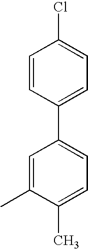 | H | logP = 4.99^{a)} |
| 144 | 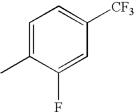 | 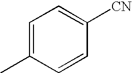 | H | logP = 6.09^{a)} |
| 145 | 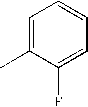 | 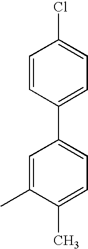 | H | logP = 4.94^{a)} |
| 146 | 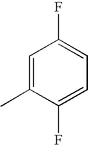 | 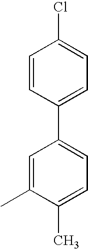 | H | logP = 4.51^{a)} |
| 147 | C₄H₉-t | 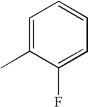 | H | m.p.: 118° C. |
| 148 | 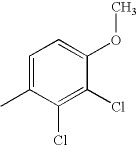 | 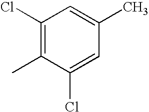 | H | |
| 149 | 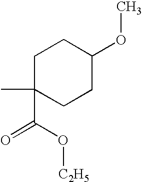 | 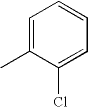 | H | logP = 2.76^{a)} |
| 150 | 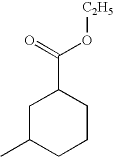 | 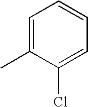 | H | logP = 3.01^{a)} |
| 151 | 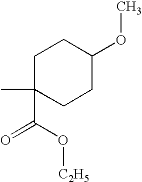 | 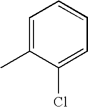 | H | logP = 2.53^{a)} |
| 152 | 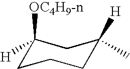 | 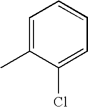 | H | logP = 3.70^{a)} |
| 153 | 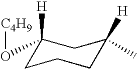 | 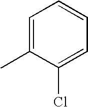 | H | logP = 4.09^{a)} |
| 154 | 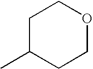 | 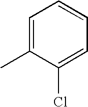 | H | logP = 1.94^{a)} |
| 155 | 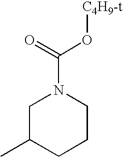 | 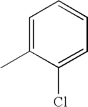 | H | logP = 3.18^{a)} |
| 156 | 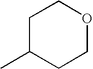 | 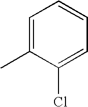 | CO—C₄H₉-t | logP = 3.87 ^{a)} |
| 157 | 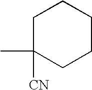 | 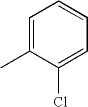 | H | logP = 2.72^{a)} |
| 158 | 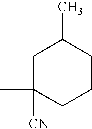 | 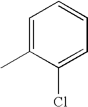 | H | logP = 3.06^{a)} |
| 159 | 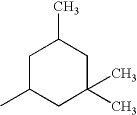 | 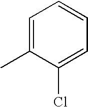 | H | logP = 4.18^{a)} |
| 160 | 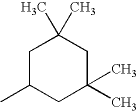 | 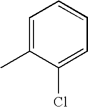 | H | logP = 4.41^{a)} |
| 161 | 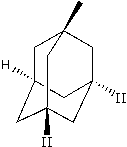 | 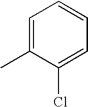 | H | logP = 4.20^{a)} |
| 162 | 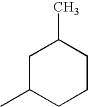 | 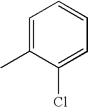 | H | logP = 3.57^{a)} |
| 163 | 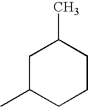 | 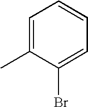 | H | logP = 3.61^{a)} |
| 164 | 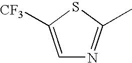 | 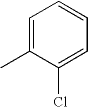 | H | logP = 3.14^{a)} |
| 165 | 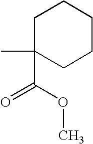 | 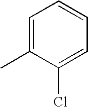 | H | logP = 3.10^{a)} |
| 166 | 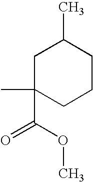 | 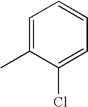 | H | logP = 3.48^{a)} |
| 167 | 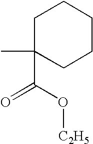 | 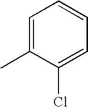 | H | logP = 3.40^{a)} |
| 168 | 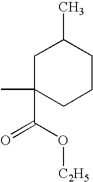 | 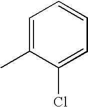 | H | logP = 3.78^{a)} |
| 169 | 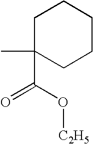 | 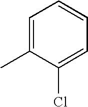 | CO—C₄H₉-t | logP = 5.50^{a)} |
| 170 | 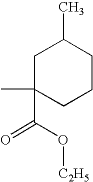 | 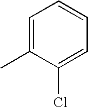 | CO—C₄H₉-t | logP = 5.85^{a)} |
| 171 | 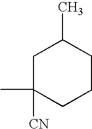 | 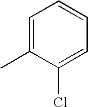 | CO—C₄H₉-t | logP = 5.01^{a)} |
| 172 | 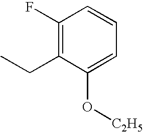 | 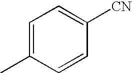 | H | logP = 2.61^{a)} |
| 173 | 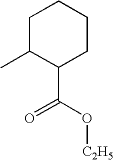 | 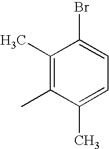 | H | logP = 3.90^{a)} |
| 174 | 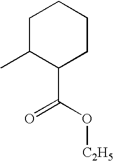 | 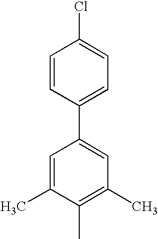 | H | logP = 4.87^{a)} |
| 175 | 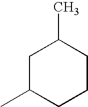 | 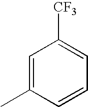 | H | logP = 4.56^{a)} |
| 176 | 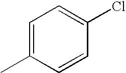 | 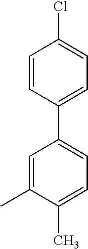 | H | |
| 177 | 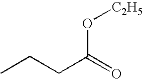 | 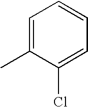 | H | logP = 2.21^{a)} |
| 178 | 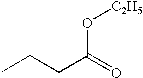 | 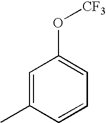 | H | logP = 3.95^{a)} |
| 179 | 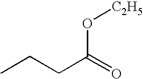 | 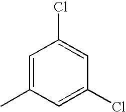 | H | logP = 2.80^{a)} |
| 180 | 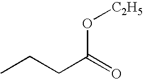 | 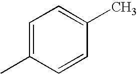 | H | logP = 2.74^{a)} |
| 181 | 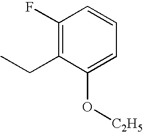 | 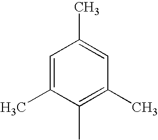 | H | logP = 3.68^{a)} |
| 182 | 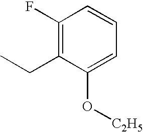 | 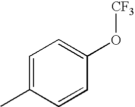 | H | logP = 4.13^{a)} |
| 183 | 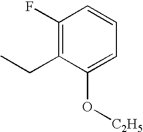 | 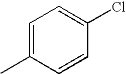 | H | logP = 3.85^{a)} |
| 184 | 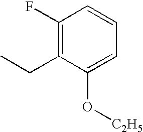 | 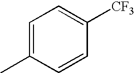 | H | logP = 3.81^{a)} |
| 185 | 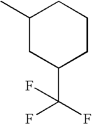 | 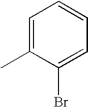 | CO—C₄H₉-t | logP = 5.34^{a)} |
| 186 | 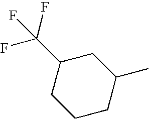 | 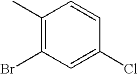 | H | logP = 2.53^{a)} |
| 187 | 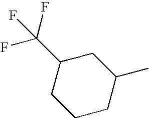 | 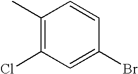 | H | logP = 2.47^{a)} |
| 188 | 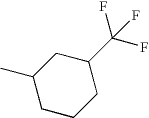 | 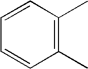 | H | logP = 3.11^{a)} |
| 189 | 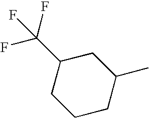 | 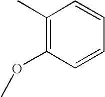 | H | logP = 3.32^{a)} |
| 190 | 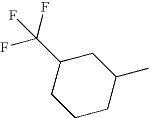 | 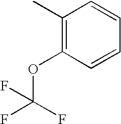 | H | logP = 3.77^{a)} |
| 191 | 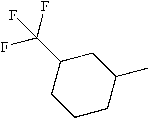 | 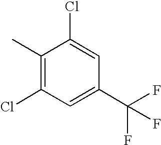 | H | logP = 4.05^{a)} |
| 192 | 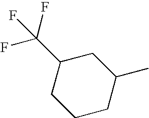 | 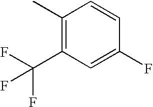 | H | logP = 3.56^{a)} |
| 193 | 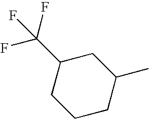 | 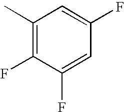 | H | logP = 3.42^{a)} |
| 194 | 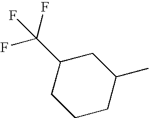 | 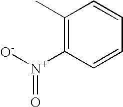 | H | logP = 3.01^{a)} |
| 195 | 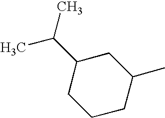 | 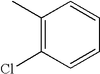 | H | |
| 196 | 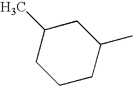 | 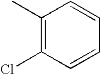 | H | logP = 3.57^{a)} |
| 197 | 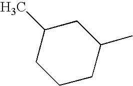 | 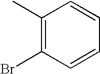 | H | logP = 3.61^{a)} |
| 198 | 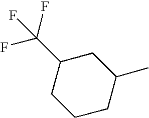 | 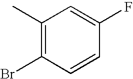 | H | logP = 3.46^{a)} |
| 199 | 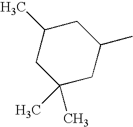 | 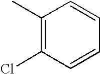 | H | logP = 4.18^{a)} |
| 200 | 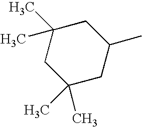 | 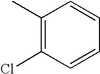 | H | logP = 4.41^{a)} |
| 201 | 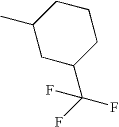 | 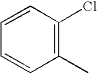 | CO—C₄H₉-t | logP = 5.29^{a)} |
| 202 | 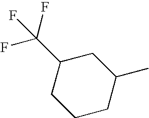 | 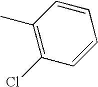 | CH₂OC₂H₅ | logP = 4.53^{a)} |
| 203 | 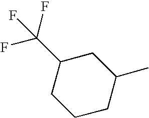 | 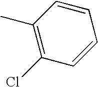 | 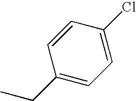 | |
| 204 | 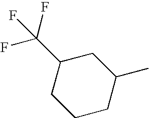 | 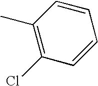 | 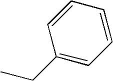 | logP = 5.01^{a)} |
| 205 | 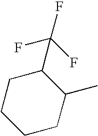 | 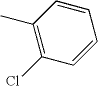 | H | logP = 3.27^{a)} |
| 206 | 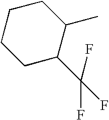 | 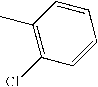 | CO—C₄H₉-t | logP = 5.22^{a)} |
| 207 | 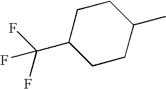 | 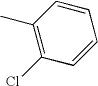 | CO—C₄H₉-t | logP = 5.31^{a)} |
| 208 | 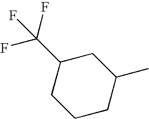 | 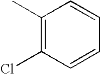 | 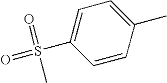 | logP = 4.89^{a)} |
| 209 | 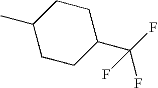 | 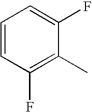 | H | |
| 210 | 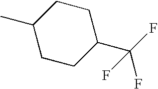 | 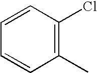 | H | logP = 3.59^{a)} |
| 211 | 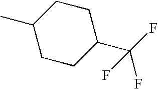 | 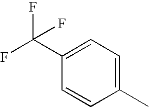 | H | logP = 4.28^{a)} |
| 212 | 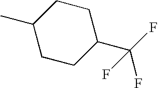 | 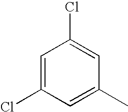 | H | logP = 4.48^{a)} |
| 213 | 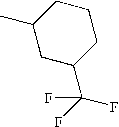 | 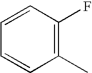 | H | logP = 3.42^{a)} |
| 214 | 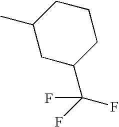 | 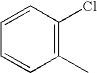 | H | logP = 3.53^{a)} |
| 215 | 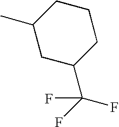 | 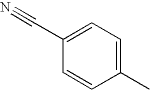 | H | logP = 3.04^{a)} |
| 216 | 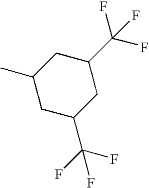 | 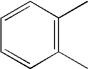 | H | logP = 3.85^{a)} |
| 217 | 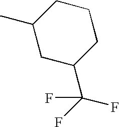 | 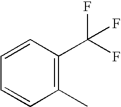 | H | |
| 218 | 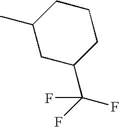 | 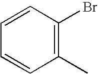 | H | |
| 219 | 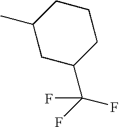 | 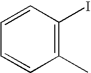 | H | |
| 220 | 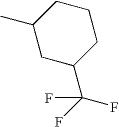 | 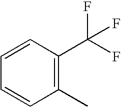 | CO—C₄H₉-t | |
| 221 | 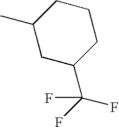 | 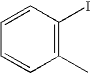 | CO—C₄H₉-t | |

a) The logP values given in table 1 were determined in accordance with EEC Directive 79/831 Annex V.A8 by HPLC (High Performance Liquid Chromatography) on a reversed-phase column (C 18). Temperature: 43° C.

(a) Mobile phases for the determination in the acid range: 0.1% aqueous phosphoric acid, acetonitrile; linear gradient from 10% acetonitrile to 90% acetonitrile—corresponding measurement results are marked a) in table 1.

(b) Mobile phases for the determination in the neutral range: 0.01 molar aqueous phosphate buffer solution, acetonitrile; linear gradient from 10% acetonitrile to 90% acetonitrile—corresponding measurement results are marked b) in table 1.

Calibration was carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

### Example A

Phaedon larvae test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Cabbage leaves (Brassica oleracea) are treated by being dipped into the active compound preparation of the desired concentration and populated with larvae of the mustard beetle (Phaedon cochleariae) while the leaves are still moist.

After the desired period of time, the kill in % is determined. 100% means that all beetle larvae have been killed; 0% means that none of the beetle larvae have been killed.

In this test, for example, the compounds of Preparation Examples 4, 5, 6, 7, 8, 9, 209, 210, 211, 211, 212, 213, 214 and 216 show good activity.

TABLE A
Plant-damaging insects
Phaedon test

| Active compounds/ | Active compound | Kill rate |
|---|---|---|
| Example No. | concentration in ppm | in % after 7^{d} |
| (4) | 1000 | 100 |
| (5) | 1000 | 100 |
| (6) | 1000 | 100 |
| (7) | 1000 | 100 |
| (8) | 500 | 100 |
| (9) | 500 | 90 |
| (209) | 500 | 100 |
| (210) | 500 | 100 |
| (211) | 500 | 90 |
| (212) | 500 | 100 |
| (213) | 500 | 100 |
| (214) | 500 | 100 |
| (216) | 500 | 100 |

### Example No. B

Phaedon test (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of Chinese cabbage (Brassica pekinensis) are sprayed with an active compound preparation of the desired concentration and, after drying, populated with larvae of the mustard beetle (Phaedon cochleariae).

After the desired period of time, the effect in % is determined. 100% means that all beetle larvae have been killed; 0% means that none of the beetle larvae have been killed.

In this test, for example, the Preparation Examples 195, 196, 197, 186, 187, 189, 190, 198, 199, 200, 218, 217, 191, 192, 193, 194, 212, 214, 205, 207, 203, 204, 221, 201, 220, 185, 219, 202, 208 and 188 show good activity.

TABLE B
Plant-damaging insects
Phaedon test (spray treatment)

| Active compounds/ | Active compound | Kill rate |
|---|---|---|
| Example No. | concentration in g/ha | in % after 7^{d} |
| (195) | 500 | 100 |
| (196) | 500 | 100 |
| (197) | 500 | 100 |
| (186) | 500 | 100 |
| (187) | 500 | 100 |
| (189) | 500 | 83 |
| (190) | 500 | 100 |
| (198) | 500 | 100 |
| (199) | 500 | 100 |
| (200) | 500 | 100 |
| (218) | 500 | 100 |
| (217) | 500 | 100 |
| (191) | 500 | 100 |
| (192) | 500 | 100 |
| (193) | 500 | 100 |
| (194) | 500 | 100 |
| (212) | 500 | 100 |
| (214) | 100 | 100 |
| (205) | 500 | 100 |
| (207) | 500 | 100 |
| (203) | 500 | 100 |
| (204) | 500 | 100 |
| (221) | 500 | 100 |
| (201) | 500 | 100 |
| (220) | 500 | 100 |
| (185) | 500 | 100 |
| (219) | 500 | 100 |
| (202) | 500 | 100 |
| (208) | 500 | 100 |
| (188) | 500 | 100 |

### Example C

Spodoptera frugiperda test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amount of solvent and the stated amount of emulsifier, and the concentrate is diluted with emulsifier-containing water to the desired concentration.

Cabbage leaves (Brassica oleracea) are treated by being dipped into the active compound preparation of the desired concentration and populated with caterpillars of the army worm (Spodoptera frugiperda) while the leaves are still moist.

After the desired period of time, the kill in % is determined. 100% means that all caterpillars have been killed; 0% means that none of the caterpillars have been killed.

In this test, for example, the compounds of the Preparation Examples 10, 11, 12; 13, 212, 214 and 215 show good activity.

TABLE C
Plant-damaging insects
Spodoptera frugiperda test

| Active compounds/ | Active compound | Kill rate |
|---|---|---|
| Example No. | concentration in ppm | in % after 7^{d} |
| (10) | 500 | 100 |
| (11) | 500 | 100 |
| (12) | 500 | 100 |
| (13) | 500 | 100 |
| (212) | 500 | 100 |
| (214) | 500 | 100 |
| (215) | 500 | 85 |

### Example D

Spodoptera frugiperda test

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of corn leaves (Zea mays) are sprayed with an active compound preparation of the desired concentration and, after drying, populated with caterpillars of the army worm (Spodoptera frugiperda).

After the desired period of time, the effect in % is determined. 100% means that all caterpillars have been killed; 0% means that none of the caterpillars have been killed.

In this test, for example, the Preparation Examples 195, 196, 197, 186, 190, 218, 217, 212, 203, 204, 201, 185, 219, 202 and 188 show good activity.

TABLE D
Plant-damaging insects
Spodoptera frugiperda test

| Active compounds/ | Active compound | Kill rate |
|---|---|---|
| Example No. | concentration in g/ha | in % after 7^{d} |
| (195) | 500 | 100 |
| (196) | 500 | 100 |
| (197) | 500 | 100 |
| (186) | 500 | 100 |
| (190) | 500 | 83 |
| (218) | 500 | 100 |
| (217) | 500 | 100 |
| (212) | 500 | 100 |
| (203) | 500 | 100 |
| (204) | 500 | 100 |
| (201) | 500 | 100 |
| (185) | 500 | 100 |
| (219) | 500 | 100 |
| (202) | 500 | 100 |
| (188) | 500 | 100 |

### Example E

Meloidogyne test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

Vessels are filled with sand, active compound solution, Meloidogyne incognita egg/larvae suspension and lettuce seeds. The lettuce seeds germinate and the plants develop. On the roots, galls are formed.

After the desired period of time, the nematicidal effect is determined in % by means of gall formation. 100% means that no galls were found; 0% means that the number of galls on the treated plants corresponds to that of the untreated control.

In this test, for example, the compounds of Preparation Examples 10, 215 and 220 show good activity.

TABLE E
Plant-damaging nematodes
Meloidogyne test

| Active compounds/ | Active compound | Kill rate |
|---|---|---|
| Example No. | concentration in ppm | in % after 14^{d} |
| (215) | 20 | 80 |
| (220) | 20 | 80 |
| (10) | 20 | 99 |

### Example F

Myzus test (spray treatment, Brassica pekinensis)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of Chinese cabbage (Brassica pekinensis) which are infested by all stages of the green peach aphid (Myzus persicae) are sprayed with an active compound preparation of the desired concentration.

After the desired period of time, the effect in % is determined. 100% means that all aphids have been killed; 0% means that none of the aphids have been killed.

In this test, for example, the compounds of Preparation Examples 2, 3, 4, 217, 218, 206, 207, 221, 201, 220, 185, 219 and 202 show good activity.

TABLE F
Plant-damaging insects
Myzus test (spray treatment)

| Active compounds/ | Active compound | Kill rate |
|---|---|---|
| Example No. | concentration in g/ha | in % after 5^{d} |
| (3) | 500 | 90 |
| (2) | 500 | 90 |
| (4) | 500 | 70 |
| (218) | 500 | 80 |
| (217) | 500 | 100 |
| (206) | 500 | 90 |
| (207) | 500 | 100 |
| (221) | 500 | 100 |
| (201) | 500 | 100 |
| (220) | 500 | 100 |
| (185) | 500 | 100 |
| (219) | 500 | 90 |
| (202) | 500 | 80 |

### Example G

Plutella test

| | |
|---|---|
| Solvent: | 7 parts by weight of dimethylformamide |
| Emulsifier: | 2 parts by weight of alkylaryl polyglyol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the active compound preparation of the desired concentration and populated with caterpillars of the diamond back moth (Plutella xylostella) while the leaves are still moist.

After the desired period of time, the kill in % is determined. 100% means that all caterpillars have been killed; 0% means that none of the caterpillars have been killed.

In this test, for example, the compounds of Preparation Examples 3 and 214 show good activity.

TABLE G
Plant-damaging insects
Plutella test

| Active compounds/ | Active compound | Kill rate |
|---|---|---|
| Example No. | concentration in ppm | in % after 7^{d} |
| (3) | 1000 | 90 |
| (214) | 500 | 100 |

### Example H

Plutella test; resistant strain

| | |
|---|---|
| Solvent: | 7 parts by weight of dimethylformamide |
| Emulsifier: | 2 parts by weight of alkylaryl polyglyol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the active compound preparation of the desired concentration and populated with caterpillars of the diamond back moth (Plutella xylostella, resistant strain) while the leaves are still moist.

After the desired period of time, the kill in % is determined. 100% means that all caterpillars have been killed; 0% means that none of the caterpillars have been killed.

In this test, for example, the compound of Preparation Example 214 shows good activity.

TABELLE H
Plant-damaging insects
Plutella test, resistant strain

| | Active compound | Kill rate |
|---|---|---|
| Active compounds/Example No. | concentration in ppm | in % after 7^{d} |
| (214) | 500 | 100 |

### Example I

Aphis fabae test

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Shoots of young broadbeans (Vicia faba) which are heavily infested by the black bean aphid (Aphis fabae) are treated by being dipped into the active compound preparation of the desired concentration.

After the desired period of time, the kill in % is determined. 100% means that all aphids have been killed; 0% means that none of the aphids have been killed.

In this test, for example, the compound of Preparation Example 4 shows good activity.

TABLE I
Plant-damaging insects
Aphis fabae test

| | Active compound | Kill rate |
|---|---|---|
| Active compounds/Example No. | concentration in ppm | in % after 6^{d} |
| (4) | 1000 | 99 |

### Example J

Tetranychus test

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Disks of beans (Phaseolus vulgaris), which are infested by all stages of the common spidermite (Tetranychus urticae) are sprayed with an active compound preparation of the desired concentration.

After the desired period of time, the effect in % is determined. 100% means that all spidermites have been killed; 0% means that none of the spidermites have been killed.

In this test, for example, the compounds of Preparation Examples 191, 192, 193, 214207, 221, 201, 185 and 202 show good activity.

TABLE J
Plant-damaging insects
Tetranychus test

| Active compounds/ | Active compound | Kill rate |
|---|---|---|
| Example No. | concentration in ppm | in % after 5^{d} |
| (191) | 500 | 90 |
| (192) | 500 | 90 |
| (193) | 500 | 90 |
| (214) | 500 | 90 |
| (207) | 500 | 90 |
| (221) | 500 | 80 |
| (201) | 500 | 100 |
| (185) | 500 | 80 |
| (202) | 500 | 80 |

### Example K

Spodoptera exigua test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the active compound preparation of the desired concentration and populated with caterpillars of the army worm (Spodoptera exigua) while the leaves are still moist.

After the desired period of time, the kill in % is determined. 100% means that all caterpillars have been killed; 0% means that none of the caterpillars have been killed.

In this test, for example, the compound of Preparation Example 214 shows good activity.

TABLE K
Plant-damaging insects
Spodoptera exigua test

| Active compounds/ | Active compound | Kill rate |
|---|---|---|
| Example No. | concentration in ppm | in % after 7^{d} |
| (214) | 500 | 100 |

### Example L

Myzus test - oral

| | |
|---|---|
| Solvent: | 80 parts by weight of acetone |

To produce a suitable preparation of active compound, 1 part by weight of active compound is mixed with the stated amounts of solvent, and the concentrate is diluted with water to the desired concentration.

Vessels are populated with all stages of the green peach aphid (Myzus persicae), which are treated by sucking on the active compound preparation of the desired concentration.

After the desired period of time, the effect in % is determined. 100% means that all aphids have been killed; 0% means that none of the aphids have been killed.

In this test, for example, the compounds of Preparation Examples 195, 196, 217, 192, 201, 185 and 219 show good activity.

TABLE L
Plant-damaging insects
Myzus test (oral)

| | Active compound | Kill rate |
|---|---|---|
| Active compounds/Example No. | concentration in ppm | in % after 6^{d} |
| (195) | 100 | 100 |
| (196) | 100 | 100 |
| (217) | 100 | 100 |
| (201) | 20 | 100 |
| (205) | 100 | 100 |
| (185) | 20 | 100 |
| (219) | 100 | 100 |

### Example M

Myzus test (spray treatment, Brassica oleracea)

| | |
|---|---|
| Solvent: | 7 parts by weight of dimethylformamide |
| Emulsifier: | 10 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) which are heavily infested by the green peach aphid (Myzus persicae) are treated by spraying with the active compound preparation at the desired concentration.

After the desired period of time, the kill in % is determined. 100% means that all animals have been killed; 0% means that none of the animals have been killed.

In this test, for example, the compounds of Preparation Examples 201 and 185 show good activity.

TABLE M
Plant-damaging insects
Myzus test (spray treatment)

| | Active compound | Kill rate |
|---|---|---|
| Active compounds/Example No. | concentration in g/ha | in % after 7^{d} |
| (201) | 500 | 98 |
| (185) | 500 | 95 |

### Example N

Aphis gossypii test

| | |
|---|---|
| Solvent: | 7 parts by weight of dimethylformamide |
| Emulsifier: | 10 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cotton leaves (Gossypium hirsutum) which are heavily infested by the cotton aphid (Aphis gossypii) are treated by spraying with the active compound preparation at the desired concentration.

After the desired period of time, the kill in % is determined. 100% means that all animals have been killed; 0% means that none of the animals have been killed.

In this test, for example, the compounds of Preparation Examples 201 and 185 show good activity.

TABLE N
Plant-damaging insects
Aphis gossypii test

| | Active compound | Kill rate |
|---|---|---|
| Active compounds/Example No. | concentration in g/ha | in % after 7^{d} |
| (201) | 500 | 99 |
| (185) | 500 | 95 |

### Example O

Critical concentration test/Soil insects - treatment of transgenic plants

| | |
|---|---|
| Test insect: | Diabrotica balteata - larvae in soil |
| Solvent: | 7 parts by weight of acetone |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of active compound in the preparation is virtually immaterial, only the amount by weight of active compound per volume unit of soil, which is stated in ppm (mg/l) matters. The soil is filled into 0.251 pots, and these are allowed to stand at 20° C.

Immediately after the preparation, 5 pregerminated maize corns of the cultivar YIELD GUARD (trademark of Monsanto Comp., USA) are placed into each pot. After 2 days, the appropriate test insects are placed into the treated soil. After a further 7 days, the efficacy of the active compound is determined by counting the maize plants that have emerged (1 plant=20% activity).

### Example P

Heliothis virescens test - Treatment of transgenic plants

| | |
|---|---|
| Solvent: | 7 parts by weight of acetone |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soybean shoots (Glycine max) of the cultivar Roundup Ready (trademark of Monsanto Comp. USA) are treated by being dipped into the preparation of active compound of the desired concentration and are populated with the tobacco budworm Heliothis virescens while the leaves are still moist.

After the desired period of time, the kill of the insects is determined.

1. A method of controlling animal pests, comprising contacting a compound of the formula (I) with pests or their habitat, said compound of the formula (I) having the structure: 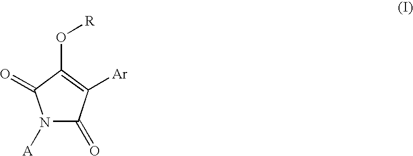
wherein
A represents optionally substituted alkyl, alkenyl, alkynyl, cycloalkyl, cycloalkenyl, cycloalkylalkyl, aryl, arylalkyl, heterocyclyl or heterocyclylalkyl, or
A represents adamantyl,
Ar represents substituted aryl, and
R represents hydrogen or one of the groupings below 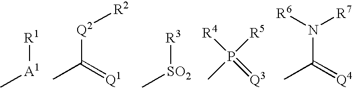
where
A¹ represents a carbonyl group (C═O) or represents methylene,
Q¹, Q², Q³ and Q⁴ each represent O (oxygen) or S (sulfur),
R¹ represents optionally substituted alkyl, alkenyl, alkynyl, cycloalkyl, cycloalkylalkyl, aryl, arylalkyl, heterocyclyl or heterocyclylalkyl when A¹ represents a carbonyl group and, when A¹ represents methylene, R¹ represents optionally substituted alkyl, alkenyl, alkynyl, cycloalkyl, cycloalkylalkyl, aryl, arylalkyl, heterocyclyl, heterocyclylalkyl, alkoxy, alkylthio, alkylsulfinyl, alkylsulfonyl, alkylamino, dialkylamino, alkylcarbonyl-amino, N-alkyl-alkylcarbonylamino, alkoxycarbonylamino, N-alkyl-alkoxycarbonylamino, alkenyloxy, alkynyloxy, alkenylamino, alkynylamino, N-alkyl-alkenylamino, N-alkyl-alkynylamino, cycloalkyloxy, cycloalkylalkoxy, cycloalkylamino, cycloalkylalkylamino, N-alkyl-cycloalkylamino, N-alkyl-cyclo-alkylalkylamino, aryloxy, arylalkoxy, arylamino, arylalkylamino, N-alkyl-arylamino or N-alkyl-arylalkylamino,
R² represents optionally substituted alkyl, alkenyl, alkynyl, cycloalkyl, cycloalkylalkyl, aryl, arylalkyl, heterocyclyl or heterocyclylalkyl,
R³ represents optionally substituted alkyl, alkenyl, alkynyl, cycloalkyl, cycloalkylalkyl, aryl, arylalkyl, heterocyclyl or heterocyclylalkyl,
R⁴ and R⁵ are identical or different and independently of one another represent optionally substituted alkyl, alkoxy, alkylthio, alkylamino, dialkylamino, phenyl, phenoxy or phenylthio,
R⁶ represents hydrogen or optionally substituted alkyl or alkenyl, and
R⁷ represents hydrogen or optionally substituted alkyl, alkoxy, alkenyl, cycloalkyl, cycloalkylalkyl, aryl or arylalkyl, or R⁷ together with R⁶ represents optionally substituted alkanediyl in which optionally one methylene group is replaced by oxygen or sulfur,
or a salt thereof formed by a reaction with a basic compound.

2. The method as claimed in claim 1 in which
A represents optionally cyano-, halogen-, C₁-C₆-alkoxy-, C₁-C₆-alkylthio-, C₁-C₆-alkylsulfinyl-, C₁-C₆-alkylsulfonyl- or C₁-C₆-alkoxy-carbonyl-substituted C₁-C₈-alkyl, optionally cyano- or halogen-substituted C₂-C₈-alkenyl or C₂-C₈-alkynyl, optionally cyano-, halogen-, C₁-C₆-alkyl-, C₁-C₆-haloalkyl-, C₁-C₆-alkoxy- or C₁-C₆-haloalkoxy-substituted C₃-C₈-cycloalkyl, C₄-C₈-cycloalkenyl or C₃-C₈-cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl or phenyl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl- or C₁-C₃-alkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group, or
A represents C₁-C₆-alkoxy-carbonyl-substituted C₃-C₈-cycloalkyl, C₄-C₈-cycloalkenyl, C₃-C₈-cycloalkyl-C₁-C₄-alkyl or adamantyl or optionally cyano-, halogen-, C₁-C₆-alkyl-, C₁-C₆-haloalkyl-, C₁-C₆-alkoxy- or C₁-C₆-haloalkoxy-substituted adamantyl,
Ar represents phenyl substituted with one to five identical or different substituents,
R represents hydrogen or one of the groupings below 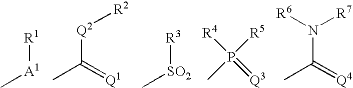
where
A¹ represents a carbonyl group (C═O) or represents methylene,
Q¹, Q², Q³ and Q⁴ each represent O (oxygen) or S (sulfur),
R¹ represents optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted C₁-C₁₀ alkyl, optionally cyano-, halogen- or C₁-C₆-alkoxy-carbonyl-substituted C₂-C₁₀ alkenyl or C₂-C₁₀ alkynyl optionally cyano-, halogen-, C₁-C₆-alkyl- or C₁-C₆-haloalkyl-substituted C₃-C₆ cycloalkyl or C₃-C₆ cycloalkyl-C₁-C₄-alkyl optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄ alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group when A¹ is a carbonyl group and, when A¹ is methylene, R¹ represents optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted C₁-C₁₀ alkyl, optionally cyano-, halogen- or C₁-C₆-alkoxy-carbonyl-substituted C₂-C₁₀ alkenyl or C₂-C₁₀ alkynyl, optionally cyano-, halogen-, C₁-C₆-alkyl- or C₁-C₆-haloalkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group, optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted C₁-C₁₀ alkoxy, C₁-C₁₀ alkylthio, C₁-C₁₀ alkylsulfinyl, C₁-C₁₀ alkylsulfonyl, C₁-C₁₀ alkylamino, di(C₁-C₁₀)alkylamino, C₁-C₁₀ alkylcarbonylamino, N—(C₁-C₁₀)alkyl-(C₁-C₁₀)alkylcarbonylamino, C₁-C₁₀) alkoxycarbonylamino or N—(C₁-C₁₀)alkyl-(C₁-C₁₀)alkoxycarbonylamino, optionally cyano-, halogen- or C₁-C₆-alkoxy-carbonyl-substituted C₂-C₁₀ alkenyloxy, C₂-C₁₀ alkynyloxy, C₂-C₁₀ alkenylamino, C₁-C₁₀ alkynylamino, N—(C₁-C₆-alkyl)-(C₂-C₁₀)alkenylamino or N—(C₁-C₆-alkyl)-(C₂-C₁₀)alkynylamino, optionally cyano-, halogen-, C₁-C₆-alkyl- or C₁-C₆-haloalkyl-substituted C₃-C₆ cycloalkyloxy, C₃-C₆ cycloalkyl-C₁-C₄-alkoxy, C₃-C₆ cycloalkylamino, C₃-C₆ cycloalkyl-C₁-C₄-alkylamino, N—(C₁-C₆-alkyl)-C₃-C₆-cycloalkylamino or N—(C₁-C₆-alkyl)-(C₃-C₆)cycloalkyl(C₁-C₄)alkylamino, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryloxy, C₆-C₁₀ aryl-C₁-C₄-alkoxy, C₆-C₁₀ arylamino, C₆-C₁₀ aryl(C₁-C₄)alkylamino, N—(C₁-C₄)alkyl-(C₆-C₁₀)arylamino or N—(C₁-C₄)alkyl-(C₆-C₁₀)aryl(C₁-C₄)alkylamino,
R² represents optionally cyano-, halogen- or C₁-C₆-alkoxy-substituted C₁-C₆ alkyl, optionally cyano- or halogen-substituted C₃-C₆ alkenyl or C₃-C₆ alkynyl, optionally cyano-, halogen- or C₁-C₆-alkyl-substituted C₃-C₆ cycloalkyl or C₃-C₆ cycloalkyl-C₁-C₄-alkyl optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R³ represents optionally halogen-substituted C₁-C₁₀ alkyl, optionally halogen-substituted C₂-C₁₀ alkenyl or C₂-C₁₀ alkynyl, optionally halogen-, C₁-C₆-alkyl- or C₁-C₆-haloalkyl-substituted C₃-C₆ cycloalkyl or C₃-C₆ cycloalkyl(C₁-C₄)alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R⁴ and R⁵ are identical or different and independently of one another represent optionally cyano-, halogen- or C₁-C₆-alkoxy-substituted C₁-C₆ alkyl, C₁-C₆ alkoxy, C₁-C₆ alkylthio, C₁-C₆ alkylamino or di(C₁-C₆)alkylamino, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl, phenoxy or phenylthio,
R⁶ represents hydrogen, optionally cyano-, halogen- or C₁-C₆-alkoxy-substituted C₁-C₆ alkyl or optionally cyano- or halogen-substituted C₂-C₆ alkenyl, and
R⁷ represents hydrogen, optionally cyano-, halogen- or C₁-C₆-alkoxy-substituted C₁-C₆ alkyl or C₁-C₆ alkoxy, optionally cyano- or halogen-substituted C₂-C₆ alkenyl, optionally cyano-, halogen- or C₁-C₆-alkyl-substituted C₃-C₆ cycloalkyl or C₃-C₆ cycloalkyl(C₁-C₄)alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or R⁷ together with R⁶ represents optionally hydroxyl-, cyano- or C₁-C₆-alkyl-substituted C₂-C₆-alkanediyl in which optionally one methylene group is replaced by O (oxygen) or S (sulfur), or a salt thereof formed by a reaction with a basic compound.

3. The method as claimed in claim 1 in which
A represents optionally cyano-, halogen-, C₁-C₆-alkoxy-, C₁-C₆-alkylthio-, C₁-C₆-alkylsulfinyl-, C₁-C₆-alkylsulfonyl- or C₁-C₆-alkoxy-carbonyl-substituted C₁-C₈-alkyl, optionally cyano- or halogen-substituted C₂-C₈-alkenyl or C₂-C₈-alkynyl, optionally cyano-, halogen-, C₁-C₆-alkyl-, C₁-C₆-haloalkyl-, C₁-C₆-alkoxy- or C₁-C₆-haloalkoxy-substituted C₃-C₈-cycloalkyl, C₄-C₈-cycloalkenyl or C₃-C₈-cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl or phenyl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl- or C₁-C₃-alkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
Ar represents phenyl substituted with one to five identical or different substituents,
R represents hydrogen or one of the groupings below 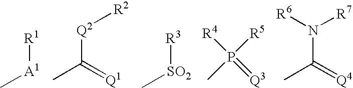
where
A¹ represents a carbonyl group (C═O) or methylene,
Q¹, Q², Q³ and Q⁴ each represent O (oxygen) or S (sulfur),
R¹ represents optionally cyano-, halogen-, C₁-C₆-alkoxy-, C₁-C₆-alkylthio-, C₁-C₆-alkylsulfinyl-, C₁-C₆-alkylsulfonyl-, C₁-C₆-al ylamino- or di(C₁-C₆-alkyl)amino-substituted C₁-C₁₀ alkyl, optionally cyano-, halogen- or C₁-C₆-alkoxy-carbonyl-substituted C₂-C₁₀ alkenyl or C₂-C₁₀ alkynyl, optionally cyano-, halogen-, C₁-C₆-alkyl- or C₁-C₆-haloalkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group when A¹ is a carbonyl group and, when A¹ is methylene, R¹ represents optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted C₁-C₁₀ alkyl, optionally cyano-, halogen- or C₁-C₆-alkoxy-carbonyl-substituted C₂-C₁₀ alkenyl or C₂-C₁₀ alkynyl, optionally cyano-, halogen-, C₁-C₆-alkyl- or C₁-C₆-haloalkyl-substituted C₃-C₆ cycloalkyl or C₃-C₆ cycloalkyl-C₁-C₄-alkyl optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S), optionally cyano-, halogen-, C₁-C₆-alkoxy-, C₁-C₆-alkylthio-, C₁-C₆-alkylsulfinyl-, C₁-C₆-alkylsulfonyl-, C₁-C₆-alkylamino- or di(C₁-C₆-alkyl)amino-substituted C₁-C₁₀ alkoxy, C₁-C₁₀ alkylthio, C₁-C₁₀ alkylsulfinyl, C₁-C₁₀ alkylsulfonyl, C₁-C₁₀ alkylamino, di(C₁-C₁₀)alkylamino, C₁-C₁₀ alkylcarbonylamino, N—(C₁-C₁₀)alkyl-C₁-C₁₀-alkylcarbonylamino, C₁-C₁₀ alkoxycarbonylamino or N—(C₁-C₁₀)alkyl-C₁-C₁₀-alkoxycarbonylamino, optionally cyano-, halogen- or C₁-C₆-alkoxy-carbonyl-substituted C₂-C₁₀ alkenyloxy, C₂-C₁₀ alkynyloxy, C₂-C₁₀ alkenylamino, C₂-C₁₀ alkynylamino, N—(C₁-C₆-alkyl)-C₂-C₁₀-alkenylamino or N—(C₁-C₆-alkyl)-C₂-C₁₀-alkynylamino, optionally cyano-, halogen-, C₁-C₆-alkyl- or C₁-C₆-haloalkyl-substituted C₃-C₆ cycloalkyloxy, C₃-C₆ cycloalkyl(C₁-C₄)alkoxy, C₃-C₆ cycloalkylamino, C₃-C₆ cycloalkyl(C₁-C₄)alkylamino, N—(C₁-C₆-alkyl)-C₃-C₆-cycloalkylamino or N—(C₁-C₆-alkyl)-C₃-C₆-cycloalkyl(C₁-C₄)alkylamino, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryloxy, C₆-C₁₀ aryl(C₁-C₄)alkoxy, C₆-C₁₀ arylamino, C₆-C₁₀ aryl(C₁-C₄)alkylamino, N—(C₁-C₄)alkyl-C₆-C₁₀-arylamino or N—(C₁-C₄)alkyl-C₆-C₁₀-aryl(C₁-C₄)alkylamino,
R² represents optionally cyano-, halogen- or C₁-C₆-alkoxy-substituted C₁-C₆ alkyl, optionally cyano- or halogen-substituted C₃-C₆ alkenyl or C₃-C₆, optionally cyano-, halogen- or C₁-C₆-alkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group optionally one (C═O) group, and optionally one (C═S) group,
R³ represents optionally halogen-substituted C₁-C₁₀ alkyl, optionally halogen-substituted C₂-C₁₀ alkenyl or C₂-C₁₀ alkynyl, represents in each case optionally halogen-, C₁-C₆-alkyl- or C₁-C₆-haloalkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R⁴ and R⁵ are identical or different and independently of one another represent optionally cyano-, halogen- or C₁-C₆-alkoxy-substituted C₁-C₆ alkyl, C₁-C₆ alkoxy, C₁-C₆ alkylthio, C₁-C₆ alkylamino or di(C₁-C₆)alkylamino, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl, phenoxy or phenylthio,
R⁶ represents hydrogen, optionally cyano-, halogen- or C₁-C₆-alkoxy-substituted C₁-C₆ alkyl or optionally cyano- or halogen-substituted C₂-C₆ alkenyl, and
R⁷ represents hydrogen, optionally cyano-, halogen- or C₁-C₆-alkoxy-substituted C₁-C₆ alkyl or C₁-C₆ alkoxy, optionally cyano- or halogen-substituted C₂-C₆ alkenyl, optionally cyano-, halogen- or C₁-C₆-alkyl-substituted C₃-C₆ cycloalkyl or C₃-C₆ cycloalkyl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or R⁷ together with R⁶ represents optionally hydroxyl-, cyano- or C₁-C₆-alkyl-substituted C₂-C₆-alkanediyl in which optionally one methylene group is replaced by O (oxygen) or S (sulfur),
or a salt thereof formed by a reaction with a basic compound.

4. A compound of the formula (IA) 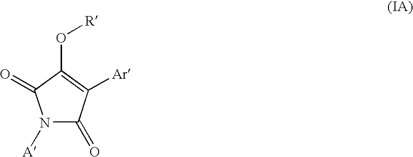
in which
A′ represents optionally cyano-, halogen-, C₁-C₅-alkoxy-, C₁-C₅-alkylthio-, C₁-C₅-alkylsulfinyl-, C₁-C₅-alkylsulfonyl- or C₁-C₅-alkoxy-carbonyl-substituted C₁-C₆-alkyl, optionally cyano- or halogen-substituted C₂-C₆-alkenyl or C₂-C₆-alkynyl, optionally cyano-, halogen-, C₁-C₄-alkyl-, C₁-C₄-haloalkyl-, C₁-C₄-alkoxy- or C₁-C₄-haloalkoxy-substituted C₃-C₅-cycloalkyl, C₇-C₈-cycloalkyl, C₅-C₆-cycloalkenyl or C₃-C₇-cycloalkyl-C₁-C₃-alkyl, optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl- or C₁-C₃-alkylenedioxy-substituted phenyl or phenyl-C₁-C₃-alkyl, optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl- or C₁-C₃-alkylenedioxy-substituted heterocyclyl or heterocyclyl-C₁-C₄-alkyl, where the heterocyclyl group of said heterocyclyl or heterocyclyl-C₁-C₄-alkyl contains 1 to 5 carbon atoms, 1 to 3 nitrogen atoms, optionally 1 oxygen atom, optionally 1 sulfur atom, optionally one (SO) group, optionally one (SO₂) group, optionally one (C═O) group, and optionally one (C═S) group, or the cyclohexyl group below 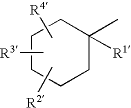
in which
R^{1′} represents hydrogen, cyano, C₁-C₅-alkyl or C₁-C₅-alkoxy-carbonyl,
R^{2′}, R^{3′} and R^{4′} are identical or different and individually represent hydrogen, halogen, C₁-C₅-alkyl, C₁-C₅-haloalkyl, C₁-C₅-alkoxy, C₁-C₅-haloalkoxy or benzyloxy, or two of R^{2′}, R^{3′} and R^{4′}, when adjacent to each other, together represent C₃-C₅-alkanediyl which is optionally interrupted by 1 or 2 heteroatoms selected from the group consisting of N, O, and S or
A′ represents cyano-, halogen-, C₁-C₄-alkyl-, C₁-C₄-haloalkyl-, C₁-C₄-alkoxy- or C₁-C₄-haloalkoxy-substituted adamantyl or represents the cyclohexyl group below 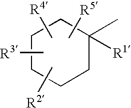
in which
R^{1′} represents hydrogen, cyano, C₁-C₅-alkyl or C₁-C₅-alkoxy-carbonyl,
R^{2′}, R^{3′} R^{4′}, and R^{5′} are identical or different and individually represent hydrogen, halogen, C₁-C₅-alkyl, C₁-C₅-haloalkyl, C₁-C₅-alkoxy, C₁-C₅-haloalkoxy, C₁-C₅-alkoxy-carbonyl or benzyloxy, or two of R^{2′}, R^{3′}, R^{4′}, and R^{5′}, when adjacent to each other, together represent C₃-C₅-alkanediyl which is optionally interrupted by 1 or 2 heteroatoms selected from the group consisting of N, O and S.
Ar′ represents phenyl substituted by one to five substituents selected from the group consisting of
nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, phenyl substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenoxy optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenyl-C₁-C₃-alkyl optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenyl-C₁-C₃-alkoxy optionally substituted with one or more substituents selected from the group consisting of nitro, cyano, carbamoyl, thiocarbamoyl, halogen C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenyl-C₁-C₃-alkylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyl optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyloxy optionally substituted with one or more substituents selected from the group consisting of nitro, cyano, carbamoyl, thiocarbamoyl, halogen, C₁-C₅-alkyl, C₁-C₅-haloalkyl, C₂-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyl-C₁-C₃-alkyl optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyl-C₁-C₃-alkoxy optionally substituted with one or more substituents selected from the group consisting of nitro, cyano, carbamoyl, thiocarbamoyl, halogen, C₁-C₅-alkyl, C₁-C₅-haloalkyl. C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, and heterocyclyl-C₁-C₃-alkylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, where the heterocyclyl group of said heterocyclyl, heterocyclyloxy, heterocyclylthio, heterocyclyl-C₁-C₃-alkyl, heterocyclyl-C₁-C₃-alkoxy, or heterocyclyl-C₁-C₃-alkylthio contains 1 to 5 carbon atoms, 1 to 3 nitrogen atoms, optionally 1 oxygen atom, optionally 1 sulfur atom, optionally one (SO) group, optionally one (SO₂) group, optionally one (C═O) group, and optionally one (C═S) group, provided that Ar′ is not 3,4-dimethoxyphenyl or 3,4-methylenedioxyphenyl,
R′ represents hydrogen or one of the groupings below 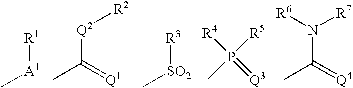
where
A¹ represents a carbonyl group (C═O) or methylene,
Q¹, Q², Q³ and Q⁴ each represent O (oxygen) or S (sulfur),
R¹ represents optionally cyano-, halogen-, C₁-C₆-alkoxy-, C₁-C₆-alkylthio-, C₁-C₆-alkylsulfinyl- or C₁-C₆-alkylsulfonyl-substituted C₁-C₁₀ alkyl, optionally cyano- or halogen-substituted C₂-C₁₀ alkenyl or C₂-C₁₀ alkynyl, optionally cyano-, halogen-, C₁-C₆-alkyl- or C₁-C₆-haloalkyl-substituted C₃-C₆ cycloalkyl or C₃-C₆ cycloalkyl-C₁-C₄-alkyl optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally additionally one (C═O) group, and optionally one (C═S) group,
R² represents optionally cyano-, halogen- or C₁-C₆-alkoxy-substituted C₁-C₆ alkyl, optionally cyano- or halogen-substituted C₃-C₆ alkenyl or C₃-C₆ alkynyl, optionally cyano-, halogen- or C₁-C₆-alkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R³ represents optionally halogen-substituted C₁-C₁₀ alkyl, optionally halogen-substituted C₂-C₁₀ alkenyl or C₂-C₁₀ alkynyl, optionally halogen-, C₁-C₆-alkyl- or C₁-C₆-haloalkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R⁴ and R⁵ are identical or different and independently of one another represent optionally cyano-, halogen- or C₁-C₆-alkoxy-substituted C₁-C₆ alkyl, C₁-C₆ alkoxy, C₁-C₆ alkylthio, C₁-C₆ alkylamino or di(C₁-C₆)alkylamino, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl, phenoxy or phenylthio,
R⁶ represents hydrogen, optionally cyano-, halogen- or C₁-C₆-alkoxy-substituted C₁-C₆ alkyl or optionally cyano- or halogen-substituted C₂-C₆ alkenyl, and
R⁷ represents hydrogen, optionally cyano-, halogen- or C₁-C₆-alkoxy-substituted C₁-C₆ alkyl or C₁-C₆ alkoxy, optionally cyano- or halogen-substituted C₂-C₆ alkenyl, optionally cyano-, halogen- or C₁-C₆-alkyl-substituted C₃-C₆ cycloalkyl or C₃-C₆ cycloalkyl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or R⁷ together with R⁶ represents optionally hydroxyl-, cyano- or C₁-C₆-alkyl-substituted C₂-C₆-alkanediyl in which optionally one methylene grouping is replaced by O (oxygen) or S (sulfur),
or a salt thereof formed by a reaction with a basic compound.

5. The compound of the formula (IA) as claimed in claim 4 in which
A′ represents optionally cyano-, halogen-, C₁-C₅-alkoxy-, C₁-C₅-alkylthio-, C₁-C₅-alkylsulfinyl-, C₁-C₅-alkylsulfonyl- or C₁-C₅-alkoxy-carbonyl-substituted C₁-C₆-alkyl, optionally cyano- or halogen-substituted C₂-C₆-alkenyl or C₂-C₆-alkynyl, optionally cyano-, halogen-, C₁-C₄-alkyl-, C₁-C₄-haloalkyl-, C₁-C₄-alkoxy- or C₁-C₄-haloalkoxy-substituted C₃-C₅-cycloalkyl, C₇-C₈-cycloalkyl, C₅-C₆-cycloalkenyl or C₃-C₇-cycloalkyl-C₁-C₃-alkyl, optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl- or C₁-C₃-alkylenedioxy-substituted phenyl or phenyl-C₁-C₃-alkyl, optionally nitro-, cyano-, halogen-, C₁-C₅-alkyl-, C₁-C₅-haloalkyl-, C₁-C₅-alkoxy-, C₁-C₅-haloalkoxy-, C₁-C₅-alkylthio-, C₁-C₅-haloalkylthio-, C₁-C₅-alkoxy-carbonyl- or C₁-C₃-alkylenedioxy-substituted heterocyclyl or heterocyclyl-C₁-C₄-alkyl, where the heterocyclyl group of said heterocyclyl or heterocyclyl-C₁-C₄-alkyl contains 1 to 5 carbon atoms, 1 to 3 nitrogen atoms, optionally 1 oxygen atom, optionally 1 sulfur atom, optionally one (SO) group, optionally one (SO₂) group, optionally one (C═O) group, and optionally one (C═S) group, or represents the cyclohexyl group below 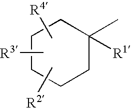
in which
R^{1′} represents hydrogen, cyano, C₁-C₅-alkyl or C₁-C₅-alkoxy-carbonyl,
R^{2′}, R^{3′} and R^{4′} are identical or different and individually represent hydrogen, halogen, C₁-C₅-alkyl, C₁-C₅-haloalkyl, C₁-C₅-alkoxy, C₁-C₅-haloalkoxy or benzyloxy, or two of R^{2′}, R^{3′} and R^{4′}, when adjacent to each other, together represent C₃-C₅-alkanediyl which is optionally interrupted by 1 or 2 heteroatoms selected from the group consisting of N, O and S,
Ar′ represents phenyl, substituted with one to five substituents selected from the group consisting of:
nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, phenyl substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenoxy optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, phenyl-C₁-C₃-alkyl optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅ haloalkylsulfonyl, phenyl-C₁-C₃-alkoxy substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅ haloalkylthio, C₁-C₅-alkylsulfinyl, C₁-C₅-haloalkylsulfinyl, C₁-C₅-alkylsulfonyl, and C₁-C₅-haloalkylsulfonyl, phenyl-C₁-C₃-alkylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyl optionally substituted with one or more substituents selected from the group consisting of nitro, cyano, carbamoyl, thiocarbamoyl, halogen, C₁-C₅-alkyl, C₁-C₅-haloalkyl C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinlyl, C₁-C₅-haloalkylsulfinyl, C₁-C₅ alkylsulfonyl, and C₁-C₅-haloalkylsulfonyl, heterocyclyloxy optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyl-C₁-C₃-alkyl optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, heterocyclyl-C₁-C₃-alkoxy optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, and heterocyclyl-C₁-C₃-alkylthio optionally substituted with one or more substituents selected from the group consisting of nitro, cyano, carbamoyl, thiocarbamoyl, halogen, C₁-C₅-alkyl, C₁-C₅-haloalkyl, C₂-C₅-alkoxy, C₁-C₅-haloalkoxy, C₁-C₃-alkoxy-C₁-C₃-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, and C₁-C₅-haloalkylsulfonyl, where the heterocyclyl group of said heterocyclyl, heterocyclyloxy, heterocyclylthio, heterocyclyl-C₁-C₃-alkyl, heterocyclyl-C₁-C₃-alkoxy, or heterocyclyl-C₁-C₃-alkylthio contains 1 to 5 carbon atoms, 1 to 3 nitrogen atoms, optionally 1 oxygen atom, optionally 1 sulfur atom, optionally one (SO) group, optionally one (SO₂) group, optionally one (C═O) group, and optionally one (C═S) group, provided that Ar′ is not 3,4-dimethoxyphenyl or 3,4-methylenedioxyphenyl,
R′ represents hydrogen or one of the groupings below 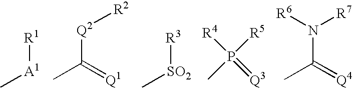
where
A¹ represents a carbonyl group (C═O) or methylene,
Q¹, Q², Q³ and Q⁴ each represent O (oxygen) or S (sulfur),
R¹ represents optionally cyano-, halogen-, C₁-C₆-alkoxy-, C₁-C₆-alkylthio-, C₁-C₆-alkylsulfinyl- or C₁-C₆-alkylsulfonyl-substituted C₁-C₁₀ alkyl, optionally cyano- or halogen-substituted C₂-C₁₀ alkenyl or C₂-C₁₀ alkynyl, optionally cyano-, halogen-, C₁-C₆-alkyl- or C₁-C₆-haloalkyl-substituted C₃-C₆ cycloalkyl or C₃-C₆ cycloalkyl-C₁-C₆-alkyl optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl has 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R² represents optionally cyano-, halogen- or C₁-C₆-alkoxy-substituted C₁-C₆ alkyl, optionally cyano- or halogen-substituted C₃-C₆ alkenyl or C₃-C₆ alkynyl, optionally cyano-, halogen- or C₁-C₆-alkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R³ represents optionally halogen-substituted C₁-C₁₀ alkyl, optionally halogen-substituted C₂-C₁₀ alkenyl or C₂-C₁₀ alkynyl, optionally halogen-, C₁-C₆-alkyl- or C₁-C₆-haloalkyl-substituted C₃-C₆ cycloalkyl or C₃-C₆ cycloalkyl-C₁-C₄-alkyl, optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted heterocyclyl or heterocyclyl-C₁-C₄-alkyl, where the heterocyclyl group of said heterocyclyl or heterocyclyl-C₁-C₄-alkyl contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group,
R⁴ and R⁵ are identical or different and independently of one another represent optionally cyano-, halogen- or C₁-C₆-alkoxy-substituted C₁-C₆ alkyl, C₁-C₆ alkoxy, C₁-C₆ alkylthio, C₁-C₆ alkylamino or di(C₁-C₆)alkylamino, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted phenyl, phenoxy or phenylthio,
R⁶ represents hydrogen, optionally cyano-, halogen- or C₁-C₆-alkoxy-substituted C₁-C₆ alkyl or represents optionally cyano- or halogen-substituted C₂-C₆ alkenyl, and
R⁷ represents hydrogen, optionally cyano-, halogen- or C₁-C₆-alkoxy-substituted C₁-C₆ alkyl or C₁-C₆ alkoxy, optionally cyano- or halogen-substituted C₂-C₆ alkenyl, optionally cyano-, halogen- or C₁-C₆-alkyl-substituted C₃-C₆ cycloalkyl or C₃-C₆ cycloalkyl-C₁-C₄-alkyl, or optionally nitro-, cyano-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkoxy-carbonyl-, C₁-C₃-alkylenedioxy- or C₁-C₃-haloalkylenedioxy-substituted C₆-C₁₀ aryl or C₆-C₁₀ aryl-C₁-C₄-alkyl, or R⁷ together with R⁶ represents optionally hydroxyl-, cyano- or C₁-C₆-alkyl-substituted C₂-C₆-alkanediyl in which optionally one methylene group is replaced by O (oxygen) or S (sulfur),
or a salt thereof formed by a reaction with a basic compound.

6. A process for preparing a compound of the formula (IA) as claimed in claim 4, comprising reacting a compound of the formula (II) 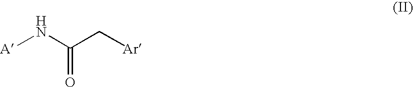
in which
A′ and Ar′ are as defined in claim 4
with a compound of the formula (III) 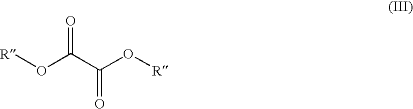
in which
R″ represents alkyl,
optionally in the presence of a reaction auxiliary and optionally in the presence of a diluent,
to give a compound of the formula (IB) 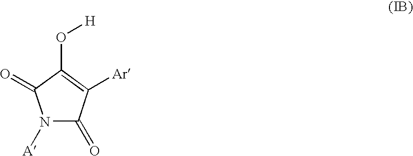
and optionally reacting the compound of the formula (IB) with a compound of the formula (IV)
X—R′ (IV)
in which
R′ is as defined in claim 4 and
X represents halogen,
optionally in the presence of a reaction auxiliary and optionally in the presence of a diluent.

7. A pesticide preparation, comprising at least one compound of the formula (IA) as claimed in claim 4.

8. A method for controlling animal pests, comprising contacting a compound of the formula (IA) as claimed in claim 4 with pests or their habitat.

9. (canceled)

10. A process for preparing a pesticide preparation, comprising mixing a compound of the formula (IA) as claimed in claim 4 with one or more extenders or surfactants or a combination thereof.

11. The method of claim 2, wherein Ar represents phenyl substituted with one to five identical or different substituents selected from the group consisting of:
nitro, cyano, carbamoyl, thiocarbamoyl, halogen, C₁-C₆-alkyl, C₁-C₆-haloalkyl, C₁-C₆-alkoxy, C₁-C₆-haloalkoxy, C₁-C₄-alkoxy-C₁-C₄-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, phenyl, phenoxy, phenylthio, phenyl-C₁-C₄-alkyl, phenyl-C₁-C₄-alkoxy, phenyl-C₁-C₄-alkylthio, heterocyclyl, heterocyclyloxy, heterocyclylthio, heterocyclyl-C₁-C₄-alkyl, heterocyclyl-C₁-C₄-alkoxy, and heterocyclyl-C₁-C₄-alkylthio, where the phenyl group of said phenyl, phenoxy, phenylthio, phenyl-C₁-C₄-alkyl, phenyl-C₁-C₄-alkoxy, and phenyl-C₁-C₄-alkylthio is optionally substituted with one or more substituents selected from the group consisting of nitro, cyano, carbamoyl, thiocarbamoyl, halogen, C₁-C₆-alkyl, C₁-C₆-haloalkyl, C₁-C₆-alkoxy, C₁-C₆-haloalkoxy, C₁-C₄-alkoxy-C₁-C₄-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, and C₁-C₆-haloalkylsulfonyl, and where the heterocyclyl group of said heterocyclyl, heterocyclyloxy, heterocyclylthio, heterocyclyl-C₁-C₄-alkyl, heterocyclyl-C₁-C₄alkoxy, or heterocyclyl-C₁-C₄-alkylthio contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group.

12. The method of claim 3, wherein Ar represents phenyl substituted with one to five identical or different substituents selected from the group consisting of:
nitro, cyano, carbamoyl, thiocarbamoyl, halogen, C₁-C₆-alkyl, C₁-C₆-haloalkyl, C₁-C₆-alkoxy, C₁-C₆-haloalkoxy, C₁-C₄-alkoxy-C₁-C₄-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, phenyl, phenoxy, phenylthio, phenyl-C₁-C₄-alkyl, phenyl-C₁-C₄-alkoxy, phenyl-C₁-C₄-alkylthio, heterocyclyl, heterocyclyloxy, heterocyclylthio, heterocyclyl-C₁-C₄-alkyl, heterocyclyl-C₁-C₄-alkoxy, heterocyclyl-C₁-C₄-alkylthio, where the phenyl group of said phenyl, phenoxy, phenylthio, phenyl-C₁-C₄-alkyl, phenyl-C₁-C₄-alkoxy, and phenyl-C₁-C₄-alkylthio is optionally substituted with one or more substituents selected from the group consisting of nitro, cyano, carbamoyl, thiocarbamoyl, halogen, C₁-C₆-alkyl, C₁-C₆-haloalkyl, C₁ C₆-alkoxy, C₁-C₆-haloalkoxy, C₁-C₄-alkoxy-C₁-C₄-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, and C₁-C₆-haloalkylsulfonyl, and where the heterocyclyl group of said heterocyclyl, heterocyclyloxy, heterocyclylthio, heterocyclyl-C₁-C₄-alkyl, heterocyclyl-C₁-C₄-alkoxy, or heterocyclyl-C₁-C₄-alkylthio contains 1 to 6 carbon atoms, 1 to 4 nitrogen atoms, optionally 1 oxygen atom, optionally 1 sulfur atom, optionally one (SO) group, optionally one (SO₂) group, optionally one (C═O) group, and optionally one (C═S) group.

13. A pesticide preparation, comprising at least one compound of the formula (I): 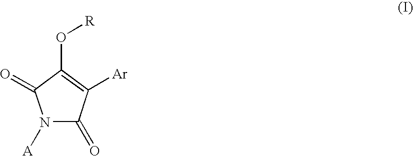
wherein
A represents optionally substituted alkyl, alkenyl, alkynyl, cycloalkyl, cycloalkenyl, cycloalkylalkyl, aryl, arylalkyl, heterocyclyl or heterocyclylalkyl, or
A represents adamantyl,
Ar represents substituted aryl, and
R represents hydrogen or one of the groupings below 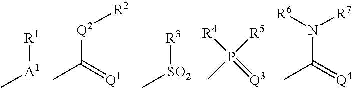
where
A¹ represents a carbonyl group (C═O) or methylene,
Q¹, Q², Q³ and Q⁴ each represent O (oxygen) or S (sulfur),
R¹ represents optionally substituted alkyl, alkenyl, alkynyl, cycloalkyl, cycloalkylalkyl, aryl, arylalkyl, heterocyclyl or heterocyclylalkyl when A¹ represents a carbonyl group and, when A¹ represents methylene, R¹ represents optionally substituted alkyl, alkenyl, alkynyl, cycloalkyl, cycloalkylalkyl, aryl, arylalkyl, heterocyclyl, heterocyclylalkyl, alkoxy, alkylthio, alkylsulfinyl, alkylsulfonyl, alkylamino, dialkylamino, alkylcarbonylamino, N-alkyl-alkylcarbonylamino, alkoxycarbonylamino, N-alkyl-alkoxycarbonylamino, alkenyloxy, alkynyloxy, alkenylamino, alkynylamino, N-alkyl-alkenylamino, N-alkyl-alkynylamino, cycloalkyloxy, cycloalkylalkoxy, cycloalkylamino, cycloalkylalkylamino, N-alkyl-cycloalkylamino, N-alkyl-cycloalkylalkylamino, aryloxy, arylalkoxy, arylamino, arylalkylamino, N-alkyl-arylamino or N-alkyl-arylalkylamino,
R² represents optionally substituted alkyl, alkenyl, alkynyl, cycloalkyl, cycloalkylalkyl, aryl, arylalkyl, heterocyclyl or heterocyclylalkyl,
R³ represents optionally substituted alkyl, alkenyl, alkynyl, cycloalkyl, cycloalkylalkyl, aryl, arylalkyl, heterocyclyl or heterocyclylalkyl,
R⁴ and R⁵ are identical or different and independently of one another represent optionally substituted alkyl, alkoxy, alkylthio, alkylamino, dialkylamino, phenyl, phenoxy or phenylthio,
R⁶ represents hydrogen or optionally substituted alkyl or alkenyl, and
R⁷ represents hydrogen or optionally substituted alkyl, alkoxy, alkenyl, cycloalkyl, cycloalkylalkyl, aryl or arylalkyl, or R⁷ together with R⁶ represents optionally substituted alkanediyl in which optionally one methylene group is replaced by oxygen or sulfur,
or a salt thereof formed by a reaction with a basic compound.

14. A process for preparing a pesticide preparation, comprising
mixing a compound of the formula (I): 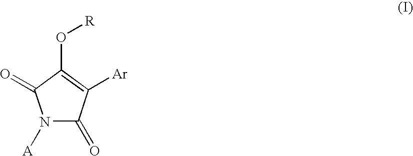
wherein
A represents optionally substituted alkyl, alkenyl, alkynyl, cycloalkyl, cycloalkenyl, cycloalkylalkyl, aryl, arylalkyl, heterocyclyl or heterocyclylalkyl, or
A represents adamantyl,
Ar represents substituted aryl, and
R represents hydrogen or one of the groupings below 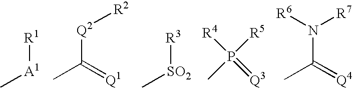
where
A¹ represents a carbonyl group (C═O) or methylene,
Q¹, Q², Q³ and Q⁴ each represent O (oxygen) or S (sulfur),
R¹ represents optionally substituted alkyl, alkenyl, alkynyl, cycloalkyl, cycloalkylalkyl, aryl, arylalkyl, heterocyclyl or heterocyclylalkyl when A¹ represents a carbonyl group and, when A¹ represents methylene, R¹ represents optionally substituted alkyl, alkenyl, alkynyl, cycloalkyl, cycloalkylalkyl, aryl, arylalkyl, heterocyclyl, heterocyclylalkyl, alkoxy, alkylthio, alkylsulfinyl, alkylsulfonyl, alkylamino, dialkylamino, alkylcarbonylamino, N-alkyl-alkylcarbonylamino, alkoxycarbonylamino, N-alkyl-alkoxycarbonylamino, alkenyloxy, alkynyloxy, alkenylamino, alkynylamino, N-alkyl-alkenylamino, N-alkyl-alkynylamino, cycloalkyloxy, cycloalkylalkoxy, cycloalkylamino, cycloalkylalkylamino, N-alkyl-cycloalkylamino, N-alkyl-cycloalkylalkylamino, aryloxy, arylalkoxy, arylamino, arylalkylamino, N-alkyl-arylamino or N-alkyl-arylalkylamino,
R² represents optionally substituted alkyl, alkenyl, alkynyl, cycloalkyl, cycloalkylalkyl, aryl, arylalkyl, heterocyclyl or heterocyclylalkyl,
R³ represents optionally substituted alkyl, alkenyl, alkynyl, cycloalkyl, cycloalkylalkyl, aryl, arylalkyl, heterocyclyl or heterocyclylalkyl,
R⁴ and R⁵ are identical or different and independently of one another represent optionally substituted alkyl, alkoxy, alkylthio, alkylamino, dialkylamino, phenyl, phenoxy or phenylthio,
R⁶ represents hydrogen or optionally substituted alkyl or alkenyl, and
R⁷ represents hydrogen or optionally substituted alkyl, alkoxy, alkenyl, cycloalkyl, cycloalkylalkyl, aryl or arylalkyl, or R⁷ together with R⁶ represents optionally substituted alkanediyl in which optionally one methylene group is replaced by oxygen or sulfur,
or a salt thereof formed by a reaction with a basic compound;
with one or more extenders or surfactants or a combination thereof.
